(19) United States
(12) Patent Application Publication
(10) Pub. No.: US 2022/0232825 A1
(43) Pub. Date: Jul. 28, 2022

(54) DEVICE AND METHOD FOR DECONTAMINATION/DISINFECTION

(71) Applicant: Aquila Bioscience Limited, Galway (IE)

(72) Inventors: Lokesh JOSHI, Galway (IE); Marta UTRATNA, Galway (IE)

(73) Assignee: Aquila Bioscience Limited, Galway (IE)

(21) Appl. No.: 17/613,645

(22) PCT Filed: May 29, 2020
(86) PCT No.: PCT/EP2020/065095

§ 371 Date: Nov. 23, 2021

(30) Foreign Application Priority Data
May 31, 2019 (GB) 1907796.5

(51) Int. Cl. A01N 25/34; A61L 2/16; A61L 2/26

(52) U.S. Cl. CPC A01N 25/34; A61L 2/26; A61L 2/16

(57) ABSTRACT

Provided are devices for viral, microbial and pathogen control and removal. A device is provided comprising a carrier material comprising a carbohydrate-based polymer, and a binding agent. The binding agent is attached by one or more covalent bonds to the carrier material, and the binding agent can bind to a target, the target being one or more of a biotoxin, a virus, a microbe, and a microbial component. Also provided are methods of using such devices for removing a biotoxin, a virus, a microbe and/or a microbial component, and processes for making a device, comprising providing a carrier material comprising a carbohydrate-based polymer, treating the carrier with an oxidising agent to produce acid and/or aldehyde groups; and contacting the treated carrier with a binding agent which comprises one or more of a lectin, a glycoprotein, and a glycoconjugate, such that the binding agent is linked to the carbohydrate-based polymer by one or more covalent bonds.

### TECHNICAL FIELD

The present invention is in the field of viral, microbial and pathogen control and removal.

### BACKGROUND

A significant problem in the modern world is how to prevent the spread of viruses, bacteria and other microbes, and their components, especially where such agents are harmful to human health or that of other organisms. As transmission of these substances can often occur through survival on and transfer between surfaces, or by passage through air or liquid, removal of viruses, microbes and microbial components from these surfaces is desirable.

Pathogenic agents are of course particularly necessary to remove, but it is also often desirable to collect or remove harmless microorganisms for various purposes, such as maintaining a sterile environment or for avoiding contamination of scientific or industrial processes.

The majority of approaches designed to remove microbial or viral load from surfaces attempt to denature or otherwise destroy the targets. For example, alcohols and other antiseptics, strong chemical disinfectants (generally oxidising agents such as bleaches, although reducing agents can be used), antibiotics, extremes of heat, and radiation such as short-wave ultraviolet light or non-thermal (cold) plasma are often used to destroy or denature viruses and microbes.

Such approaches have several major drawbacks, primarily that efficacy can vary, as microbes can have or develop resistance to almost all methods of eradication. Some microbes can produce spores which can be extremely resistant to heat, or to chemical, pharmaceutical, and ultraviolet attack, and therefore prove very difficult to destroy. Many viruses are by their nature resistant to destruction by standard means, being immune to antibacterial agents such as antibiotics, and lacking cellular processes to disrupt. Biotoxins and components of microbes or viruses are also likely to be resistant to mechanisms designed to kill living organisms, and for this reason can also be difficult to remove.

Additionally, the means used to disinfect can themselves be harmful to users. This is especially true for strong chemical disinfectants, but other means such as antibiotics can provoke allergic reactions, and use of ultraviolet light can be damaging to skin and potentially carcinogenic. It may also not be practical to apply such means in many cases, for example it is not always feasible to heat a surface to high temperatures for sterilisation.

Even if the target microbes or viruses are destroyed, harmful substances can remain, such as protein-based or non-protein-based bacterial toxins, such as lipopolysaccharides (endotoxins), or the enterotoxins, for example produced by Vibrio cholerae. Other biotoxins, such as those produced by plants and animals, present similar concerns.

Perhaps most importantly, chemical and antibiotic resistance can be evolved by microbes and retained by their descendants and even passed horizontally by gene transfer. Use of strong chemical disinfectants and hygiene products is therefore an additional risk factor, promoting mutations and making eradication procedures inefficient. Many important antimicrobial drugs, including even the strongest antibiotics and chemicals, are no longer effective, resulting in increased human (and animal) fatality rates, global epidemic threats and accelerated healthcare costs. This is equally worrying in the case of biological threat agents that in the absence of appropriate control measures can cause widespread fear and damage to human and animal lives.

There is therefore a need to produce methods and devices which are effective at removing biotoxins, viruses, microbes and microbial components, including those which may be resistant to standard methods of removal or destruction, and effectively retaining these contaminants for later analysis and/or disposal.

Existing devices which aim to remove target biotoxins, viruses, microbes and microbial components by retaining them within a material or carrier are unable to effectively retain these targets, as interactions between the biotoxins, viruses, microbes and microbial components and the material or carrier are in general insufficiently strong. For example, the removed targets may be merely adsorbed onto or into the material or carrier, for instance by hydrogen bonds or similar interactions. These approaches lead to inefficient take-up of the targets, and the release of temporarily bound targets when the material or carrier encounters another surface.

The present invention provides devices and methods for the removal of biotoxins, viruses, microbes and microbial components from contaminated surfaces and their stable retention within a carrier material.

### SUMMARY OF THE INVENTION

In a first aspect, the invention provides a device (e.g. suitably for removing a biotoxin, a virus, a microbe and/or a microbial component from a surface and/or from a gas or liquid) comprising a carrier material comprising at least one carbohydrate-based polymer, and a binding agent. The binding agent is attached by one or more covalent bonds to the carrier material, and can bind to a target, the target being one or more of a biotoxin, a virus, a microbe, and a microbial component.

The carrier material may comprise cellulose as a carbohydrate-based polymer, and may comprise one or more of cotton and paper. Where the carrier material comprises cellulose, the binding agent may be attached to the cellulose by one or more covalent bonds.

The device may comprise a fluid in which the carrier material is dissolved, suspended, dispersed, emulsified, or otherwise carried.

The binding agent may comprise one or more of an antithrombotic agent, an anti-inflammatory, an antibody, an antigen, an adhesin, an immunoglobulin, an enzyme, a hormone, a neurotransmitter, a cytokine, a protein, a globular protein, a cell attachment protein, a peptide, a cell attachment peptide, a proteoglycan, a toxin, a polysaccharide, a carbohydrate, a fatty acid, a drug, a vitamin, a DNA segment, a RNA segment, a nucleic acid, a dye and a ligand. In some embodiments, the binding agent comprises one or more of a lectin, a glycoprotein, an oligosaccharide, and a glycoconjugate.

The binding agent may comprise a lectin, which may be one or more of AIA/Jacalin, RPbAI, AAL, ABL, ACA, AMA, BPA, CAA, Calsepa, CCA, ConA, CPA, DBA, DSA, ECA, EEA, GHA, GNA, GSL-I-B4, GSL-II, HHA, HPA, Lch-A, Lch-B, LEL, LTA, MAA, MOA, MPA, NPA, PA-I, PCA, PHA-E, PHA-L, PNA, PSA, RCA-I/120, SBA, SJA, SNA-I, SNA-II, STA, UEA-I, VRA, VVA-B4, WFA, and WGA. Suitably, the lectin may be one or more of VRA, Lch-B, EEA, PA-I, PNA, CAA, GSL-I-B4, AMA, RCA-I/120, and GNA.

In some embodiments, the binding agent comprises a glycoprotein, which may be one or more of Uromodulin (Tamm-Horsfall protein), Fetuin, Asialofetuin, Invertase, Fibrinogen, alpha-1-antitrypsin, α-Crystallin, Ceruloplasmin, alpha-1-acid glycoprotein, RNAse B, Transferrin, β-lactoglobulin, C.-lactalbumin, Albumin, B-casein, C-casein, K-casein, Lactoferrin, Ovalbumin, Ovomucoid, Ovotransferrin, and derivative glycomacropeptides. Typically, the glycoprotein may be one or more of Fetuin, Asialofetuin and α-Crystallin.

In some embodiments, the binding agent is a glycoconjugate or neoglycoconjugate. The glycoconjugate or neoglycoconjugate may be one or more of Blood Group A-BSA, Blood Group B-HSA, Fuc-α-4AP-BSA, Fuc-β-4AP-BSA, 2′Fucosyllactose-BSA, Difucosyl-para-lacto-N-hexaose-APD-HSA (Lea/Lex), Tri-fucosyl-Ley-heptasaccharide-APE-HSA, Monofucosyl, monosialyllacto-N-neohexaose-APD-HSA, Gal-β-4AP-BSA, Galα1,3Gal-BSA, Gal-α-1,3Galb1, Gal-β-1,4Gal-BSA, Gal-α-1,2Gal-BSA, 4GlcNAc-HSA, Gal-α-PITC-BSA, Gal-β-ITC-BSA, Glc-β-4AP-BSA, Glc-β-ITC-BSA, GlcNAc-BSA, Globotriose-HSA, Globo-N-tetraose-APD-HSA, Globotriose-APD-HSA, GM1-pentasaccharide-APD-HSA, Asialo-GM1-tetrasaccharide-APD-HSA, Globo-N-tetraose-APD-HSA, Globotriose-APD-HSA, H Type II-APE-BSA, H-Type 2-APE-HSA, Man-α-1,3(Man-α-1,6), Man-BSA, Man-α-ITC-BSA, Man-b-4AP-BSA, LacNAc-BSA, LacNAc-α-4AP-BSA, LacNAc-β-4AP-BSA, Lac-β-4AP-BSA, Lacto-N-tetraose-APD-HSA, Lacto-N-fucopentaose I-BSA, Lacto-N-neotetraose-APD-HSA, Lacto-N-fucopentaose II-BSA, Lacto-N-fucopentaose III-BSA, Lacto-N-difucohexaose I-BSA, Lewis a-BSA, Lewis x-BSA, Lewis y-tetrasaccharide-APE-HSA, LNDI-BSA/Lewis b-BSA, Di-Lex-APE-BSA, Di-Lewisx-APE-HSA, Tri-Lex-APE-HSA, L-Rhamnose-Sp14-BSA, 3″-Sialyllactose-APD-HSA, 3′Sialyl-3-fucosyllactose-BSA, 6″-Sialyllactose-APD-HSA, Xyl-α-4AP-BSA, Xyl-β-4AP-BSA, 3′Sialyl Lewis x-BSA, 3′Sialyl Lewis a-BSA, 6-Sulfo Lewis x-BSA, 6-Sulfo Lewis a-BSA, 3-Sulfo Lewis a-BSA, 3-Sulfo Lewis x-BSA, Sialyl-LNF V-APD-HSA, Sialyl-LNnT-penta-APD-HSA.

Where the binding agent is a glycoconjugate, neoglycoconjugate or a glycoprotein, it may have terminal saccharide residues comprising one or more of mannose, N-Acetylglucosamine, N-Acetylgalactosamine, N-acetylneuraminic and N-glycolylneuraminic acid (sialic acid), Galactose, Glucose and Fucose moieties.

The carrier material may further comprise an antimicrobial substance, which may be one or more of an antiseptic, an antibiotic and a detergent. The antimicrobial substance may be silver, copper, or EDTA.

In any embodiment, the carrier material of the device may be in the form of a cloth, a wipe, a wound dressing, a swab, a filter, a pad, a blanket, a mat, a mask or a coating.

In a second aspect, the invention provides a method of removing a biotoxin, a virus, a microbe and/or a microbial component from a surface, the method comprising providing a device according to any embodiment described herein, and contacting the surface with the device.

In a third aspect, the invention provides a method of removing a biotoxin, a virus, a microbe and/or a microbial component from a gas or liquid, the method comprising providing a device according to any embodiment described herein, and passing the gas or liquid through the device.

In embodiments of either of the methods described above, the binding agent may bind to the biotoxin, the virus, the microbe and/or the microbial component to be removed. The virus, the microbe and/or the microbial component to be removed may be a spore.

In a further aspect, the invention provides a process for making a device, the method comprising providing a carrier material comprising at least one carbohydrate-based polymer, treating the carrier with an oxidising agent to produce acid and/or aldehyde groups, and contacting the treated carrier with a binding agent which comprises one or more of a lectin, a glycoprotein, and a glycoconjugate, such that the binding agent is linked to the carbohydrate-based polymer by one or more covalent bonds. The oxidising agent may be a periodate, suitably sodium periodate. The oxidising agent can be selected from the group comprising 2,2,6,6-tetramethylpiperidine-1-oxyl radical (TEMPO), sodium nitrate or sodium nitrate in phosphoric acid; activating agent tosylchloride in presence of organic solvent and a base; and combinations thereof.

The further possible features discussed in conjunction with embodiments the device according to the invention are considered to apply equally to the device made by the above process.

In a particular aspect, there is provided a device comprising a carrier material comprising cellulose; and a binding agent comprising a glycoprotein selected from one or more of: Uromodulin (Tamm-Horsfall protein), Fetuin, Asialofetuin, Invertase, Fibrinogen, alpha-1-antitrypsin, α-Crystallin, Ceruloplasmin, alpha-1-acid glycoprotein, RNAse B, Transferrin, β-lactoglobulin, C.-lactalbumin, Albumin, B-casein, C-casein, K-casein, Lactoferrin, Ovalbumin, Ovomucoid, Ovotransferrin, and derivative glycomacropeptides. The binding agent is attached by one or more covalent bonds to the cellulose, and the binding agent can bind to a target, the target being one or more of a biotoxin, a virus, a microbe, and a microbial component.

### BRIEF DESCRIPTION OF THE DRAWINGS

The invention is further illustrated by reference to the accompanying drawings in which:

FIGS. 1A to 1C show the results of efficacy tests using devices according to various embodiments of the invention for the removal of Francisella tularensis from various surfaces.

FIGS. 2A to 2D show the results of efficacy tests using devices according to various embodiments of the invention for the removal of Clostridium botulinum from various surfaces.

FIGS. 3A and 3B show the results of efficacy tests using devices according to various embodiments of the invention for the removal of Bacillus anthracis, in cell (3A) or spore (3B) forms, from various surfaces.

FIGS. 4A to 4C show the results of efficacy tests using devices according to various embodiments of the invention for the removal of influenza virus from various surfaces.

FIGS. 5A and 5B show the results of efficacy tests using devices according to various embodiments of the invention for the removal of EHEC Escherichia coli (E. coli) O157:H7 and Enterobacter cloacae from various surfaces.

FIG. 6 shows the results of efficacy tests using devices according to various embodiments of the invention for the removal of Propionibacterium acnes from plastic surfaces.

FIG. 7 shows the results of efficacy tests using devices according to an embodiment of the invention for the removal of Candida albicans from plastic surfaces at various pH levels.

FIG. 8 shows a representation of a method of testing devices according to embodiments of the invention against models of skin inoculated with various microbes.

FIGS. 9A and 9B show the results of efficacy tests using devices according to an embodiment of the invention for the removal of E. coli from porcine skin sections.

FIG. 10 shows the results of efficacy tests using devices according to an embodiment of the invention for the removal of Candida albicans from porcine skin sections.

FIG. 11 shows the results of efficacy tests using devices according to an embodiment of the invention for the removal of Aspergillus fumigatus from porcine skin sections.

### DETAILED DESCRIPTION

All references cited herein are incorporated by reference in their entirety. Unless otherwise defined, all technical and scientific terms used herein have the same meaning as commonly understood by one of ordinary skill in the art to which this invention belongs.

Prior to further setting forth the invention, a number of definitions are provided that will assist in the understanding of the invention.

As used herein, the term “target” refers to an article which it is desired to remove from a surface, or otherwise take-up into or immobilise in/on a device according to the invention. Typically, a target is a biotoxin, a virus, microbe or microbial component, and may be a pathogen. In some cases, a target can be an allergen or a microscopic component produced by non-microbial life, such as plant pollen, fungal spores, dust mite faeces and other components, potential allergens from food such as nuts and shellfish, animal or plant venoms or poisons, and animal products such as dander.

As used herein, the term “microbe” refers to a microorganism, in particular to bacteria, fungi, so-called ‘protozoa’ or any other prokaryotic or eukaryotic organism of microscopic nature.

As used herein, the term “microbial component”, “microbial product” or “microbial substance” refers to a product of a microbe which it is desired to remove from a surface, or otherwise take-up into or immobilise in/on a device according to the invention. A microbial component can be a toxin, that is, a substance that is harmful to the body, for example a protein-based or non-protein-based bacterial toxin such as lipopolysaccharide (endotoxin), or the enterotoxins, for example produced by Vibrio cholerae.

As used herein, the term “toxin” or “biotoxin” refers to a substance biological in origin that is harmful to the body. Biotoxins can be produced by microbes as discussed above, or can have other sources, such as plants or animals.

As used herein, the term “pathogen” refers to a virus or microbe which can cause a disease.

As used herein, the term “carbohydrate-based polymer” refers to a polymer which comprises monosaccharide units (simple sugar molecules) as the major or only components of its repeating polymer units. Carbohydrate-based polymers include, but are not limited to, polysaccharides as further described below, dextran, starch, glycogen, fungal beta-glucans, chitin, chitosan, cellulose and cellulose derivatives (such as cellulose acetate, celluloid, and nitrocellulose), laminarin, chrysolaminarin, xylan, arabinoxylan, mannan, fucoidan and galactomannan. While many such polymers consist only of monosaccharide units and their derivatives, copolymers comprising monosaccharides and other units exist, such as saccharide-peptide hybrid copolymers. Additionally, certain carbohydrate-based polymers, in particular where they are non-fibrous, can be dissolved, suspended, dispersed, emulsified, or otherwise carried in a fluid carrier such as a liquid or gas, as a spray, sol aerosol, emulsion, or otherwise.

As used herein, the term “polysaccharide” refers to a carbohydrate-based polymer composed of chains of monosaccharide units (simple sugar molecules), where chains can be straight or branched. Polysaccharides are commonly used to store sugars for later use, such as in the polysaccharides starch, glycogen and laminarin. Other polysaccharides can be used for structural purposes, including cellulose, fungal beta-glucans, chitin, pectins, xylans, arabinoxylans and so on. Bacteria often produce and secrete polysaccharides, for example to aid in adhesion to surfaces or to escape a host immune system.

As used herein, the term “cellulose” refers to a biological carbohydrate polymer which is made from chains of β(1→4) linked D-glucose units. Cellulose is produced by green plants for use in the cell wall, as well as other species including several algae, and some bacteria. Micronised cellulose or nano cellulose can be non-fibrous and therefore can be dissolved, suspended, dispersed, emulsified, or otherwise carried in a fluid carrier such as a liquid or gas, as a spray, sol aerosol, emulsion, or otherwise.

As used herein, the term “binding agent” refers to a biological molecule which is capable of binding to a biotoxin, a virus, microbe or microbial component. Such molecules can include one or more of an antithrombotic agent, an anti-inflammatory, an antibody, an antigen, an adhesin, an immunoglobulin, an enzyme, a hormone, a neurotransmitter, a cytokine, a protein, a globular protein, a cell attachment protein, a peptide, a cell attachment peptide, a proteoglycan, a toxin, a polysaccharide, a carbohydrate, a fatty acid, a drug, a vitamin, a DNA segment, a RNA segment, a nucleic acid, a dye and a ligand. Typically, binding agents discussed herein are one or more of a glycoprotein, an oligosaccharide, a lectin, a glycoconjugate and their derivatives.

As used herein, the term “glycoprotein” refers to proteins which have one or more oligosaccharide groups or glycans attached to them. Many secreted proteins have been ‘glycosylated’ in this way, and transmembrane proteins with an extracellular domain often have saccharide groups attached to these domains.

As used herein, the term “glycoconjugate” refers to proteins and lipids which have one or more oligosaccharide groups of glycans attached to them. Examples are glycoproteins, glycolipids, glycosphingolipids, proteoglycans and glycosaminoglycans of natural or synthetic origin. ‘Neoglycoconjugates’ or NGCs refer to artificial or synthetic glycoconjugates, in particular glycoproteins and glycolipids, where a protein or lipid backbone is chemically conjugated to one or more sugar residues. Most often proteins like bovine serum albumin (BSA) and human serum albumin (HSA) are used to prepare neoglycoconjugates.

As used herein, the term “lectin” refers to carbohydrate-binding proteins (the term carbohydrate-binding protein or CBP is used interchangeably). Lectins are specific for carbohydrate moieties such as those present on glycoproteins, glycolipids, or oligosaccharides. Some lectins are also called ‘agglutinins’, given their ability to agglutinate particles to which they bind. However, the term agglutinin can apply to any substance which allows for such agglutination, such as an antibody.

As used herein, the term “adhesin” refers to cell-surface components which are involved in the adhesion of a cell to other cells or to surfaces. These are common in pathogenic, parasitic or commensal microbes, as they are used to adhere to the host surface.

As used herein, the term “antiseptic” refers to a chemical substance that has an antimicrobial effect, in particular killing, denaturing or destroying microbes, or preventing their growth or reproduction. In general, antiseptics are safe to use on skin or living tissue, including areas like the oral cavity, but are not generally used inside the body due to efficacy or safety concerns. Some, but not all, antiseptics are effective at denaturing or destroying viruses. Many classes of antiseptics exist, including alcohols such as phenols, weak concentrations of disinfectant chemicals such as bleaches or peroxides, iodine, and some specific chemicals such as chlorhexidine gluconate and quaternary ammonium compounds.

As used herein, the term “antibiotic” refers to a chemical substance that has an antimicrobial effect which is or can be used inside the body. These substances often interfere with bacterial processes, causing the death or lysis of microbial cells, but are not generally effective against viruses or bacterial products. Many types of antibiotic are well known, such as penicillins, cephalosporins, tetracyclines, ansamycins, and so on.

The present invention describes a device and methods relating to technology for biotoxin, viral, microbial and microbe-derived component (proteins, peptides and carbohydrates) collection, decontamination/disinfection, preservation for downstream diagnostic and forensic applications and delivery. This approach targets natural binding sites of the viruses, microbes and/or their components, or biotoxins, and offers a non-toxic, environmentally friendly alternative for physical surfaces as well as biological decontamination to be used on human and animal skin and the surfaces of the mucosal epithelia. The approach is intended to be broadly specific, that is, targeting multiple kinds of pathogens for multiple purposes in multiple formats.

Cell surface protein and carbohydrate interactions are essential for the adhesion between cells. This also applies to the adhesion of certain biotoxins, viruses, microbes and proteins of microbial origin to other surfaces, such as the cells of a host organism.

Mechanisms of binding of microbes and viruses to cells, especially in the cases of commensal, symbiotic or parasitic microbes binding to host cells, are a particularly significant area of evolution. For example, host-bacteria interactions can be mediated by bacterial adhesins and their cognate glycan receptor epitopes on the host cell surfaces. The majority of adhesins on both gram-negative and gram-positive bacteria identify the suitable host via glycan markers on the epithelial cell surface of the host organisms (Kline et al, Cell Host and Microbe, 2009). Example bacterial species with their respective adhesins, target ligands and tissues can be found in Table 1. Especially in the case of pathogenic agents, the interaction between pathogen surface markers and those of the host are crucial for strong adhesion to the host, evasion of the immune system, and (in the case of intracellular pathogens and toxins) access to the cell interior. Indeed, the ability of particular bacteria to specifically adhere to host cells (often by possession of a particular surface protein or other molecule) can represent a virulence factor, making the difference between pathogenic and non-pathogenic strains. In nature, these agents are constantly being bound, retained and removed from human and animal cell surfaces before a pathogenic agent can reproduce or enter the cells to cause infections.

TABLE 1

| Bacterial Species | Adhesin Protein | Carbohydrate Ligand on Host Cell | Target Tissue |
|---|---|---|---|
| Helicobacter pylori | BabA | [Fucα2]Galβ3[Fucα4]GlcNAc (Le^{b})- | Stomach |
| Helicobacter pylori | SabA | Siaα3Galβ3[Fucα4]GlcNAc (SLe^{a})- | Stomach |
| | | Siaα3Galβ4[Fucα3]GlcNAc (SLe^{x})- | |
| Escherichia coli | FimH (Type 1 pilus) | Mannose-oligosaccharides | G.I. Tract |
| Escherichia coli | SfaS (S-pilus) | Siaα3Galβ4GlcbCer | G.I. Tract |
| Enterococcus | EfaA | D-galactose or L-fucose + glycans | G.I. Tract |
| faecalis | | | |
| Haemophilus | HifE | Sialylyganglioside-GM1 | Respiratory |
| influenzae | | | |
| Bordetella pertussis | FHA | Sulfated glycolipids, heparin | Respiratory |
| Streptococcus | Hs Antigen | α2-3-linked Sia-containing receptors | Respiratory |
| gordonii | | | |
| Strep. pneumoniae | PsaA | N-acetyl hexosamine galactose | Respiratory |

With a similar principle of carbohydrate-protein-binding technology, the current device allows for a unique approach by utilising natural and modified protein and carbohydrate epitopes being chemically attached to a carrier in a range of formats. The devices provide a multiplicity of ‘hooks’ which are able to bind to biotoxins, viruses, microbes and/or their components in competition with host attachment surfaces, and so effectively remove or trap these targets.

Therefore, in order to remove microbes and proteins of microbial origin (bacteria, viruses, phage particles, fungi and proteins from these agents), carbohydrates, proteins and protein fragments immobilised on physical material can be used to collect samples of the biotoxins, viruses, microbes and/or their components, and reduce the microbial burden on the surface, to decontaminate/disinfect the surfaces and to preserve the samples collected on the device for diagnostic and forensic purposes.

### Carrier Material

The carrier material provides a surface for the binding agent to be attached to, and a substrate in which the viral, microbial, microbe-derived component and/or biotoxin can be immobilised. Suitably, the carrier material is able to form covalent bonds with the binding agent, such that a strong and suitably irreversible connection can be made.

Typically, the carrier can comprise a carbohydrate-based polymer, suitably a polysaccharide, such as starch, glycogen, chitin, cellulose, chitin, pectins, fungal beta-glucans, xylans or arabinoxylans. The carrier can suitably comprise cellulose. For example, the carrier may comprise cellulose, hemicellulose or lignocellulose. The carrier can intrinsically contain cellulose, for example comprising plant-derived substances such as paper, cotton, viscose, linen or hemp, or a material can be blended or coated with cellulose from another source. The carrier can also comprise a blended material, such as a blend of a cellulose-containing material with a synthetic material, for example a 50% blend of cotton with polyester material. Cellulose can also be produced by microorganisms such as bacteria. In particular, bacteria of the genera Acetobacter, Sarcina ventriculi and Agrobacterium have been used to produce bacterial cellulose.

Non-fibrous cellulose based materials and carbohydrate-based polymers can serve as a carrier. In particular, micronized cellulose and nano-cellulose can serve as non-fibrous cellulose in this way. Such non-fibrous cellulose or carbohydrate-based polymers can be dissolved, suspended, dispersed, emulsified, or otherwise carried in a fluid carrier such as a liquid or gas. Therefore, when coupled to a binding agent as described herein, such preparations can be applied, for example to a recipient material, in a non-solid format such as a spray or paint.

Such non-solid formats of carbohydrate-based polymers linked to binding agents allow for a range of applications not otherwise possible. For example, such a product/device in suspension or soluble format can be sprayed or otherwise applied onto recipient materials, such as textiles, in order to impart a protective quality to said materials depending on the characteristics of the applied product. After application, the recipient material can be washed or treated, for instance with detergent or under low-pH conditions, in order to remove the previously applied product. The recipient material can then be re-treated with fresh product to regenerate the protective quality before its next use or exposure. Such an approach could be used, for example, in the treatment of personal masks or other personal protective equipment, in order to impart a protective quality against one or more target biotoxins, viruses, microbes, and/or microbial components, as appropriate for a particular purpose.

Further possible applications for non-solid formats of carbohydrate-based polymers linked to binding agents include use as a cleaning composition, which can be sprayed or otherwise applied onto a recipient material to be cleaned, and then removed together with any bound target biotoxins, viruses, microbes, and/or microbial components. Particular applications of such an approach include the washing of large shipping containers, transport hubs and hospitals, and the sterilisation of equipment for aseptic application, such as in a hospital or space context.

More generally, and for all considered formats, where it is desired to permanently kill, denature or otherwise destroy the biotoxins, viruses, microbes and/or microbial components taken up by the devices of the invention, the carrier or device can further comprise agents suitable to carry this out. For example, the carrier can be impregnated or mixed with antimicrobial or antiviral chemicals such as antibiotics, antiseptics, bleaches which may include hypochlorites, peroxides and percarbonates, and other materials with intrinsically antimicrobial properties such as silver or copper, other suitable metal ions, and metal chelating agents such as EDTA, which has been shown to have antimicrobial efficacy (Finnegan and Percival, Wound Healing Society, 2014). Other possibilities include benzoic acid, and benzalkonium chloride and other quaternary ammonium cations. Different additional substances can be chosen depending on the proposed use of the device, for example if the device is intended to be used on skin, antiseptics safe for such a use may be chosen. Stabilisers and/or preservatives may also be used, examples of which are known in the art.

In some cases, it may be desired to retain the target biotoxins, viruses, microbes and/or microbial components, for later analysis for research, diagnostic or forensic purposes. In such cases, the carrier may be substantially free of antimicrobial substances, and may even be treated to improve the likelihood of the target biotoxins, viruses, microbes and/or microbial components surviving intact for later analysis, such as by comprising buffers or other solutions, or a particular pH level, in order to support the immobilised targets. Buffers or other solutions may also be included in order to aid in the binding of the binding agents to the desired targets, as most interactions will depend on an aqueous environment, a particular pH level and so on.

It is envisioned to prepare carriers in many different forms. For example, wipes, cloth, wound dressings, filters, pads, coatings, blankets, mats, masks and other articles may be constructed of carrier material. In particular, a wipe form, similar to a tissue, towel or napkin is contemplated for use, as this provides a convenient form for wiping over a surface to be decontaminated, and for disposal afterwards. It is also contemplated that devices according to the present invention may be used for removing target biotoxins, viruses, microbes and/or microbial components from gases or liquids, for example removing airborne or aerosolised virus particles from the air. In this case, the carrier is designed to allow for the gas or liquid to be passed through, in order that the targets can be removed. As discussed above, it is also contemplated that certain carrier materials may be coupled to binding agents and dissolved, suspended, dispersed, emulsified, or otherwise carried in a fluid; accordingly, the fluids comprising such carrier materials are examples of devices according to the invention.

It may also be desirable for the devices of the present invention to be suitable for general cleaning, on living surfaces such as the skin, or non-living surfaces. As a result, it is contemplated that the carriers can comprise further components as appropriate for the intended use, such as cleansers, moisturisers, deodorants or chemicals for the removal of makeup (for example when used for the cleaning of skin), and/or detergents, scents, or cleaning agents for the removal of dust, grime, metal tarnish and the like from non-living surfaces. In this way, devices as described herein can be used for a therapeutic purpose, such as removing or killing germs from physical surfaces and from the skin (for example, to treat acne, diaper rash, and other skin conditions). In non-therapeutic uses, such as cosmetic uses, the device can be used for cleansing or moisturizing the skin, baby care, hand washing, makeup removal, or the application of deodorants.

Incontinence pads incorporating or comprising devices according to the invention are also contemplated, and configurations can be chosen which would give protection against the infectious agents which promote urinary tract diseases.

Pads and/or wipes designed for breast feeding and/or soothing of the nipples are also contemplated and can be designed to be effective in providing protection against infectious agents which cause mastitis.

### Binding Agents

Binding agents which are able to bind to target biotoxins, viruses, microbes and/or microbial components are attached to the carrier material. Any biologically-derived molecule which is capable of binding to a biotoxin, virus, microbe, or microbial component may be used in devices according to the invention. Such molecules can include one or more of an antithrombotic agent, an anti-inflammatory, an antibody, an antigen, an adhesin, an immunoglobulin, an enzyme, a hormone, a neurotransmitter, a cytokine, a protein, a globular protein, a cell attachment protein, a peptide, a cell attachment peptide, a proteoglycan, a toxin, a polysaccharide, a carbohydrate, a fatty acid, a drug, a vitamin, a DNA segment, a RNA segment, a nucleic acid, a dye and a ligand. Suitably, the binding agents comprise one or more of a glycoprotein, an oligosaccharide, a lectin and a glycoconjugate.

Binding agents suitable for use in the present invention have many origins, with many able to be derived from naturally produced solutions such as milk, urine, mucus, saliva, eggs, fungal, algal and plant extracts. Binding agents can also be synthetically produced (for example by in-vitro translation), or engineered (for example by recombinant engineering), or naturally produced binding agents can be processed or modified to make specific peptides, glycopeptides, fragments, glycans or similar, for instance to represent only the binding portion of a particular larger molecule.

Another advantage of many of the binding agents discussed herein is that they are non-toxic and environmentally friendly compared to antimicrobial or antiviral reagents commonly used. For example, polyguanidine compounds often used as biocides are in a category of compounds that are being restricted by the FDA due to toxicity to humans and cause environmental damage. Another guanidine example is chlorhexidine gluconate, where plans exist to restrict this compound to prescription-only use in the future. Similarly, quaternary ammonium compounds, such as polyionenes, are commonly used in wipes and hand sanitisers, but again the FDA is in the process of restricting their use.

While some potential binding agents can be expected to bind very specifically to only one target (in particular, antibodies), many have a broader range of potential binding targets, and can be used for the removal of more than one target biotoxin, virus, microbe and/or microbial component. However, in order to increase the number of potential targets and thereby improve the use of the device, more than one type of binding agent can be used in a device, which can be from the same class of molecule (for example, more than one species of glycoprotein) or different classes (for example, glycoproteins and lectins). As a result, depending on the desired application, a device according to the invention can be engineered to have very specific binding targets, for example in a research or forensic setting, or more general usage, as might be suitable in a domestic or field context.

Many interactions between biotoxins, viruses, microbes and/or microbial components and host cells, or the surfaces to which they adhere, are driven by carbohydrates, glycoproteins and lectins (carbohydrate binding proteins or CBPs, glycan binding proteins or GBPs) and their fragments (such as peptides). For example, the type 1 fimbrial FimH adhesin which is possessed by certain bacteria such as some E. coli strains can bind to host cell surface markers, such as CD48, TLR4 or more generally, to mannose residues, through its lectin (carbohydrate binding) domain.

Glycoconjugates including glycoproteins, glycolipids, glycosphingolipids, proteoglycans and glycosaminoglycans of natural or synthetic origin, are therefore considered for use in providing binding sites for the adhesion of target biotoxins, viruses, microbes and/or microbial components to the device. Glycoconjugates for use in devices according to the invention may have terminal residues comprising one or more of mannose, N-Acetylglucosamine, N-Acetylgalactosamine, N-acetylneuraminic and N-glycolylneuraminic acid (sialic acid), Galactose, Glucose and Fucose moieties

Suitable glycoconjugates and neoglycoconjugates for use in devices according to the invention include Blood Group A-BSA, Blood Group B-HSA, Fuc-α-4AP-BSA, Fuc-β-4AP-BSA, 2′Fucosyllactose-BSA, Difucosyl-para-lacto-N-hexaose-APD-HSA (Lea/Lex), Tri-fucosyl-Ley-heptasaccharide-APE-HSA, Monofucosyl, monosialyllacto-N-neohexaose-APD-HSA, Gal-b-4AP-BSA, Galα1,3Gal-BSA, Galα1,3Galb1, Galb1,4Gal-BSA, Galα1,2Gal-BSA, 4GlcNAc-HSA, Gal-α-PITC-BSA, Gal-β-ITC-BSA, Glc-b-4AP-BSA, Glc-β-ITC-BSA, GlcNAc-BSA, Globotriose-HSA, Globo-N-tetraose-APD-HSA, Globotriose-APD-HSA, GM1-pentasaccharide-APD-HSA, Asialo-GM1-tetrasaccharide-APD-HSA, Globo-N-tetraose-APD-HSA, Globotriose-APD-HSA, H Type II-APE-BSA, H-Type 2-APE-HSA, Manα1,3(Manα1,6), Man-BSA, Man-α-ITC-BSA, Man-b-4AP-BSA, LacNAc-BSA, LacNAc-α-4AP-BSA, LacNAc-β-4AP-BSA, Lac-β-4AP-BSA, Lacto-N-tetraose-APD-HSA, Lacto-N-fucopentaose I-BSA, Lacto-N-neotetraose-APD-HSA, Lacto-N-fucopentaose II-BSA, Lacto-N-fucopentaose III-BSA, Lacto-N-difucohexaose I-BSA, Lewis a-BSA, Lewis x-BSA, Lewis y-tetrasaccharide-APE-HSA, LNDI-BSA/Lewis b-BSA, Di-Lex-APE-BSA, Di-Lewisx-APE-HSA, Tri-Lex-APE-HSA, L-Rhamnose-Sp14-BSA, 3″-Sialyllactose-APD-HSA, 3′Sialyl-3-fucosyllactose-BSA, 6″-Sialyllactose-APD-HSA, Xyl-α-4AP-BSA, Xyl-β-4AP-BSA, 3′Sialyl Lewis x-BSA, 3′Sialyl Lewis a-BSA, 6-Sulfo Lewis x-BSA, 6-Sulfo Lewis a-BSA, 3-Sulfo Lewis a-BSA, 3-Sulfo Lewis x-BSA, Sialyl-LNF V-APD-HSA, and Sialyl-LNnT-penta-APD-HSA.

Suitable glycoproteins for use in devices according to the invention may have terminal residues comprising one or more of mannose, N-Acetylglucosamine, N-Acetylgalactosamine, N-acetylneuraminic and N-glycolylneuraminic acid (sialic acid), Galactose, Glucose and Fucose moieties. Such glycoproteins and oligosaccharides may be derived from naturally produced solutions such as milk, urine, mucus, saliva, eggs, fungal, algal and plant extracts. Particular glycoproteins which are suitable for use in devices according to the invention include Uromodulin (Tamm-Horsfall protein), Fetuin, Asialofetuin, Invertase, Fibrinogen, alpha-1-antitrypsin, a-Crystallin, Ceruloplasmin, alpha-1-acid glycoprotein, RNAse B, Transferrin, B-lactoglobulin, C.-lactalbumin, Albumin, B-casein, C-casein, K-casein, Lactoferrin, Ovalbumin, Ovomucoid, Ovotransferrin, and derivative glycomacropeptides. Mucins, being high molecular weight proteins which are heavily glycosylated, and other glycoproteins found in mucus, also may be used. These components of mucus are thought to reduce the risk of infection by interfering with microbial adherence and preventing biofilm formation (Caldara et al. Current Biology, 2012).

Lectins, or carbohydrate binding proteins, are commonly involved in the binding of bacteria and viruses to their intended targets as well as inter-cellular interactions, and innate and adaptive immune responses. Lectins are present in all organisms, and play various roles in cell adhesion, immune recognition, microbial recognition (such as for pathogens and commensals), host-recognition, toxin activity, and plant protection. In the research context, many lectins can be effectively isolated from plant and fungal species and can be engineered to alter their specificity. Lectins are used to purify and characterise glycoconjugates as well as in lectin-histochemistry to stain cells, tissues and organs, in order to understand the differences in glycosylation on biological samples under different conditions.

Lectins which are contemplated for use in the present invention, and their sources, include, but are not limited to the following:

AIA, Jacalin, (Artocarpus integrifolia) Jack fruit lectin;

RPbAI, (Robinia pseudoacacia), Black locust lectin;

AAL, (Aleuria aurantia), Orange peel fungus lectin;

ABL, (Agaricus bisporus), Edible mushroom lectin;

ACA, (Amaranthus caudatus), Amaranthin lectin;

AMA, (Arum maculatum), Lords and Ladies lectin;

BPA, (Bauhinia purpurea), Camels foot tree lectin;

CAA, (Caragana arborescens), Pea tree lectin;

Calsepa, (Calystegia sepium), Bindweed lectin;

CCA, (Cancer antennarius), California crab;

ConA, (Canavalia ensiformis), Jack bean lectin;

CPA, (Cicer arietinum), Chickpea lectin;

DBA, (Dolichos biflorus), Horse gram lectin;

DSA, (Datura stramonium), Jimson weed lectin;

ECA, (Erythrina cristagalli), Cocks comb/coral tree lectin;

EEA, (Euonymous europaeus), Spindle tree lectin;

GHA, (Glechoma hederacea) Ground ivy lectin;

GNA (Galanthus nivalis), Snowdrop lectin;

GSL-I-B4, (Griffonia simplicifolia), Griffonia/Bandeiraea lectin-I;

GSL-II, (Griffonia simplicifolia), Griffonia/Bandeiraea lectin-II;

HHA, (Hippeastrum hybrid), Amaryllis agglutinin;

HPA, (Helix pomatia), Garden snail lectin;

Lch-A, (Lens culinaris), Lentil isolectin A;

Lch-B, (Lens culinaris), Lentil isolectin B;

LEL, (Lycopersicum eculentum), Tomato lectin;

LTA, (Lotus tetragonolobus), Lotus lectin;

MAA, (Maackia amurensis), Maackia agglutinin;

MOA, (Marasmius oreades), Fairy ring mushroom lectin;

MPA, (Maclura pomifera), Osage orange lectin;

NPA, (Narcissus pseudonarcissus), Daffodil lectin;

PA-I, (Pseudomonas aeruginosa), Pseudomonas lectin;

PCA, (Phaseolus coccineus), Scarlet runner bean lectin;

PHA-E, (Phaseolus vulgaris), Kidney bean erythroagglutinin;

PHA-L, (Phaseolus vulgaris), Kidney bean leukoagglutinin;

PNA, (Arachis hypogaea), Peanut lectin;

PSA, (Pisum sativum), Pea lectin;

RCA-I/120, (Ricinus communis), Castor bean lectin I;

SBA, (Glycine max), Soy bean lectin;

SJA, (Sophora japonica), Pagoda tree lectin;

SNA-I, (Sambucus nigra), Sambucus lectin-I;

SNA-II, (Sambucus nigra), Sambucus lectin-II;

STA, (Solanum tuberosum), Potato lectin;

UEA-I, (Ulex europaeus), Gorse lectin-I;

VRA, (Vigna radiate), Mung bean agglutinin;

VVA-B4, (Vicia villosa), Hairy vetch lectin;

WFA, (Wisteria floribunda), Japanese wisteria lectin; and

WGA, (Triticum vulgaris), Wheat germ agglutinin.

### Preparation

While it is in theory possible for the binding agent to be impregnated relatively simply into the carrier material, for example by soaking the carrier material in an aqueous solution comprising the binding agent, such that it is absorbed or adsorbed into or onto the carrier, the linking of the binding agent to the carrier material is suitably carried out such that a chemical bond is created between the two, particularly, a covalent bond. Such a relatively strong and permanent bond is advantageous in that the binding agents will not be lost over time, and in that target biotoxins, viruses, microbes and microbial components will be more strongly retained on the device/carrier material, and less likely to be transferred to a subsequent surface. Without wishing to be bound by theory, it is also thought that the stronger the attachment of the binding agent to the carrier material, the more likely it is that the target will be taken up and attached to the carrier, rather than the binding agent being itself removed from the carrier. Therefore, the safety, efficacy, stability and longevity of the device are improved by the formation of covalent bonds between the binding agent and the carrier material.

The binding agent or agents to be attached to the carrier material can be provided or formulated in any suitable way. For example, the binding agent can be formulated in a buffered solution, with a diluent, and/or with an excipient or stabilizing agent. The binding agent may alternatively or additionally be provided in a format comprising a pharmaceutically acceptable vehicle which may comprise one or more of solutions, rinses, shampoos, sprays, lotions, gels, foams, lubricants, creams, ointments, soaps, non-soap bars, and powders.

In order to form a covalent bond between the binding agent and the carrier material, it may be convenient to treat the carrier material chemically to provide attachment sites for the binding agent. For example, where the carrier material comprises cellulose, the cellulose can be treated to create acid and/or aldehyde functional groups, which can react with an amino group of a protein in order to create a bond. In these carrier treatment reactions, a carbohydrate ring within the cellulose is broken open by an oxidising agent. In particular, it is considered that the oxidising agent used may be a perhalogenate such as a periodate or perchlorate, a percarbonate, a permanganate, a hypochlorite, a perborate, or a peroxide. Other oxidation methods can include the use of TEMPO (2,2,6,6-tetramethylpiperidine-1-oxyl radical) to mediate oxidation of cellulose; oxidation in phosphoric acid with sodium nitrate and/or sodium nitrite; and/or treatment with activating agent tosylchloride (toluene sulfonyl chloride) in presence of organic solvent (such as acetone/dioxane) and a base (such as Pyridine or Triethylamine). Example methods are discussed, for example, in U.S. Pat. No. 5,516,673; Cumpstey I. Chemical modification of polysaccharides. ISRN Org Chem. 2013 Sep. 10; 2013:417672; Saito T, lsogai A. TEMPO-mediated oxidation of native cellulose. The effect of oxidation conditions on chemical and crystal structures of the water-insoluble fractions. Biomacromolecules. 2004; 5(5):1983-1989; and Kim U J et al., Periodate oxidation of crystalline cellulose. Biomacromolecules. 2000; 1(3):488-492.

The aim of such treatment is to introduce reactive groups into the cellulose molecule, which may be for example one or more of aldehyde, ketone, N-hydroxysuccinimide, epoxide, imidoester, anhydride, or carbonate groups. Reactions 1 and 2 show an example reaction where sodium periodate is used to create active aldehyde groups by ring opening of a cellulose beta(1-4) linked D-glucose unit (Reaction 1), followed by the attachment of a protein, such as a lectin, glycoprotein or neoglycoconjugate (Reaction 2). This reaction may be carried out in a buffered solution in the range of pH 5 to 9 (FIG. 7).

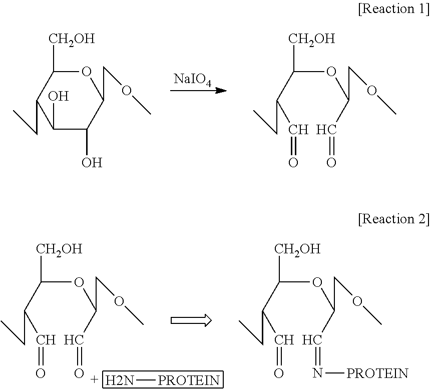

In the example given below, a ‘Schiff base’ (—CH═NH—) is created between the cellulose unit and the attached protein. Optionally, this double bond can be reduced to a single bond in order to improve the permanence of the linkage. This can be achieved by the use of a reducing agent, such as sodium cyanoborohydride. Such a reaction would also reduce the exposed CH═O moiety to CH₂OH.

By such reactions, an irreversible and highly stable link is formed, leading to the desired carbohydrates and proteins becoming embedded in the cellulosic material. Similar reactions can be used to attach glycopolymers, multimeric proteins and other biopolymers where amide or carboxylic linkage is used in the conjugation. Given that these reactions target saccharide monomers, it can be appreciated that similar approaches can be used to attach binding agents to other saccharide-containing carriers, such as polysaccharides including starch, glycogen, laminarin, fungal beta glucans, chitin, pectins, xylans, arabinoxylans, dextran or amylose. For example, dextran has been oxidised and subsequently conjugated with soy peptides (Wang and Xiong, J Food Sci Technol, 2016). Other saccharide-containing polymers such as peptidoglycan (such as that found in bacterial cell walls) could also be a substrate to which binding agents could be linked.

It can also be appreciated that methods such as those described, involving oxidation of the cellulose to which the binding agents are later bound, are preferable to methods which involve modification of the reagents to be bound themselves, as such treatment can disrupt or destroy the binding potential of the those reagents. Methods as described herein by contrast preserve the carbohydrate (or other) chemistry of the binding agents, while ensuring they are securely bound to the carrier material. Similarly, the longevity of covalent bonds is advantageous over methods relying on electrostatic attraction or other forces to bind chemicals to carrier materials, as such bonds can degrade over time.

In order to promote long term stability and sterility of the devices of the invention, the carrier materials, the carrier solutions, produced materials and/or packaging materials can be treated before, during or after the preparation steps discussed above, by a combination of filtration, heat, chemicals, irradiation and high pressure; for example by pasteurisation, autoclaving, gamma irradiation, ultraviolet radiation, electron-beam (eBeam irradiation), gas vapour sterilisation (ozone, chlorine dioxide, ethylene oxide, oxides of nitrogen) or similar approaches.

Similarly, buffers may be used to promote long term stability and sterility of the devices, such as Borate, Tris and Citrate buffers, which are further advantageous in that they are safe for ophthalmic applications.

### Target Biotoxins, Viruses, Microbes and Microbial Components

The devices according to the present invention may target biotoxins, viruses, microbes and/or microbial components which are associated with biothreat hazards, that is, potential dangers from biological weapons, synthetic biology products and/or weaponised microbes, such as in the case of bioterrorism, or potentially pandemic agents such as influenza variants, SARS, MERS, hantavirus, Nipah virus, Ebola virus, Zika virus, and so on. Devices against such targets include wipes and filters which can be used to protect against or remove these hazards. However, devices according to the invention may also be of use in research into defence against such agents, such as in general research or the production of vaccines or antitoxins. Therefore, devices as described can be used in a biosurveillance context, for instance to capture or screen for persistent biothreat agents after an intended release or during natural outbreaks, or for prophylaxis against pandemic agents and foodborne agents. As it is often vital to positively identify these agents for forensic, biosurveillance or other purposes, the fact that the devices according to the present invention do not primarily aim to destroy the targets but rather remove them is advantageous. Examples of such biothreat hazards and the species thought to cause them include bacteria such as Francisella spp. (tularemia) Bacillus anthracis (anthrax), Clostridium botulinum (botulism), Burkholderia mallei (glanders), Burkholderia pseudomallei (melioidosis); viruses such as Influenza virus, Ebola virus, Marburg virus, variola major virus (smallpox), foot-and-mouth disease virus (aphthovirus), SARS-associated coronavirus, Chapare/Lujo viruses (Arenaviridae, Q fever caused by Coxiella spp.); and toxins such as botulinum neurotoxins, ricin, abrin and shiga-like toxin. In particular, severe acute respiratory syndrome coronavirus 2 (SARS-CoV-2), also known as 2019 novel coronavirus (2019-nCoV), the strain of coronavirus that causes pandemic coronavirus disease COVID-19, is considered as a biothreat hazard which could be a target for embodiments of the present invention. Surrogate strains are species which resemble biothreat agents in one or more ways, and can be used as mimics to research various strategies to combat biothreats. Examples of species which can be used as such surrogates, and which can also be targeted by devices as described herein include Bacillus spp. (Bacillus subtilis, Bacillus atrophaeus, Bacillus mycoides); Clostridium sporogenes and Francisella tularensis subsp. holarctica LVS.

Target bacteria may also be agents causing Hospital Acquired Infections (HAI), or Health-Care Associated Infections (HCAI), or nosocomial infections, and/or antibiotic-resistant bacteria, which resist destruction by common antibiotics. Examples of such bacteria include Clostridium difficile, Methicillin-resistant Staphylococcus aureus (MRSA), Vancomycin resistant Staphylococcus aureus (VRSA), Escherichia coli (STEC, VTEC, EHEC), Clostridium difficile, Klebsiella pneumoniae, Acinetobacter baumannii, Pseudomonas aeruginosa, Enterococcus faecalis, nontuberculous mycobacteria Mycobacterium fortuitum, Proteus mirabili, and so on. An advantage of the present invention against such bacteria is that the adhesion is not affected by the mechanisms used by these bacteria to destroy or evade antibiotics.

A further advantage of the present invention is that it can be effective in the removal of bacterial spores. As previously mentioned, spores produced by some microbes are extremely resistant to heat, or to chemical, pharmaceutical, and ultraviolet attack, and therefore prove very difficult to destroy. However, the adhesion methods used by the present invention have been proven to be effective in the removal of such targets, as direct destruction of the spore is not necessarily attempted.

The targets may comprise biotoxins, viruses, microbes and microbial components which are associated with foodborne diseases. Many such targets are bacterial, such as Campylobacter spp., Clostridium spp., E. coli spp., Listeria spp., Salmonella spp., Shigella spp., Staphylococcus spp., Vibrio spp., and Helicobacter pylori. Viruses such as Norovirus (Norwalk Virus), Rotavirus, Foot-and-mouth disease virus may also be targets. In such cases, devices according to the invention may be used for the decontamination of food preparation surfaces and utensils, or may be used as mats, napkins and suchlike.

It is also contemplated that microbial components such as bacterial toxins may be targets of the devices of the present invention. Such toxins include, for example, cholera toxin, botulinum, pertussis toxin, enterotoxin, tetanus toxin, staphylococcal enterotoxins. Similarly, biotoxins of plant or animal origin, such as tetrodotoxin, ricin and abrin, are contemplated as targets of devices of the present invention. As an example, the highly toxic ricin protein is heterodimeric, with an A-chain which acts as an N-glycoside hydrolase and underlies its toxicity, and a B-chain which is a lectin which can bind galactose residues on target cell surfaces, thereby enabling cell entry. Use of devices according to the invention with particular binding agents which can interact with the B-chain lectin can be effective at removing or otherwise trapping these proteins. Some other toxic proteins such as abrin have similar lectin components and can also be targeted. In this context the present invention is advantageous compared to existing antimicrobial approaches, as toxins cannot be killed as microbes might be, and can often be resistant to denaturation by chemical or other means. The adhesive approach of the present invention allows for the removal of toxins without these problems.

Tables 2 and 3 show various bacterial toxins, and their top ten known specific interactions with lectins (Table 2), and glycoproteins/neoglycoconjugates determined based on binding analysis (Table 3). These analyses were carried out by glycan microarray, assessing the selected toxins against various lectins and glycoproteins/neoglycoconjugates. These techniques represent a tool for assessing protein—carbohydrate interactions in vitro which allows for an increase in the number of experiments possible with limited sample amounts and facilitates a profiling or screening approach prior to subsequent focused investigation. (Kilcoyne. Gerlach, Kane and Joshi, Analytical Methods, 2012).

TABLE 2

| TOP | BoNT A | BoNT B | BoNT E | BoNT F | Ricin extract | Ridin A chain |
|---|---|---|---|---|---|---|
| 1 | GSL-II | Calsepa | Calsepa | Calsepa | GSL-II | Calsepa |
| 2 | Calsepa | CCA | GNA | GSL-II | sWGA | LEL |
| 3 | AAL | PHA-L | SBA | GNA | Calsepa | DSA |
| 4 | sWGA | IPHA-E | GHA | GSL-I-B4 | DSA | GNA |
| 5 | PBS | PBS | HHA | sWGA | GNA | RCA-I/12.0 |
| 6 | PNA | GSL-II | MPA | SBA | TJA-I | NPA |
| 7 | MPA | GNA | NPA | BPA | PSA | STA |
| 8 | GNA | WFA | SJA | MPA | GHA | AAL |
| 9 | TJA-I | STA | BPA | GLS-I-A4 | CCA | HHA |
| 10 | IPSA | AAL | GLS-I-A4 | PNA | SBA | SBA |

TABLE 3

| TOP | BoNT A | BoNT B | BoNT E | BoNT F | Ricin extract | Ricin A chain |
|---|---|---|---|---|---|---|
| 1 | SLNFVHSA | SLNnTHSA | LNFPIIBSA | SLNFVHSA | SLNFVHSA | LNFPIIBSA |
| 2 | SLNnTHSA | SLNFVHSA | LNFPIBSA | SLNnTHSA | SLNnTHSA | LNFPIBSA |
| 3 | LNFPIIBSA | RB | LNDHIBSA | ASF | LNFPIIBSA | Gb4GBSA |
| 4 | LNDHIBSA | ASF | GGGNBSA | Fetuin | GlobTHSA | Ga3GBSA |
| 5 | RB | LNFPIIBSA | 2FLBSA | LNFPIIBSA | ASF | ASF |
| 6 | LNFPIBSA | MMLNnHHSA | SLNnTHSA | GlobTHSA | GlobNTHSA | Fetuin |
| 7 | ASF | LNFPIBSA | Ga3GBSA | GlobNTHSA | Fetuin | SLNnTHSA |
| 8 | 2FLBSA | GGGNBSA | BGBHSA | Xferrin | aGM1HSA | 2FLBSA |
| 9 | GGGNBSA | Gb4GBSA | SLNFVHSA | BGBHSA | DFPLNHHSA | SLNFVHSA |
| 10 | Fetuin | LNDHIBSA | DFPLNHHSA | DFPLNHHSA | LNDHIBSA | BT |

### Uses

Devices according to the invention are contemplated for use in a variety of applications, for example, prophylactically, therapeutically and topically. Possible uses include decontamination, cleaning, sample collection, sample retention, sample concentration, sample forensic analysis, wound care and healing, preventing the transmission and spread of diseases, preventing secondary contamination, preventing biofilm formation, personal hygiene, and/or lowering stress and fear associated with the risks related to transmittable agents.

Also provided are methods for treating or preventing certain conditions and ailments, using the device in accordance with the invention. These include human, animal and plant diseases, which can be mediated by bacterial, fungal, viral or toxin agents, or by eukaryotic microorganisms such as the malaria parasite, trypanosomes such as the leishmania and sleeping system parasites, and so on. For example, a target microbe or microbial component may be causing skin diseases and disorders.

Examples of skin diseases and disorders caused by fungal agents, and the species thought to underlie them include candidiasis (Candida albicans), pityriasis or tinea versicolor (Malassezia furfur or Pityrosporum orbiculare), seborrheic dermatitis (Malassezia spp.) and Athlete's foot (tinea pedis, which may be caused by fungal species including Trichophyton spp., Epidermophyton spp., and Microsporum spp.). Other infections and the agents thought to cause or be involved in them include acne vulgaris (Propionibacterium acnes, Propionibacterium granulosum and Pseudomonas aeruginosa), staphylococcal scalded skin syndrome, impetigo, ecthyma, folliculitis, furuncles, pyoderma carbuncles (Staphylococcus aureus, Streptococcus pyogenes, Pseudomonas aeruginosa), and common body odour (Propionibacterium avidum). Also considered is the treatment of mammary gland tissue inflammation (mastitis) such as mediated by Staphylococcus aureus, Streptococcus agalactiae, Streptococcus bovis, Escherichia coli, Pseudomonas aeruginosa, Streptococcus uberis and Staphylococcus chromogenem. Devices can be produced which aim to remove these agents from the skin to aid in treatment, or from surfaces in order to reduce transmission. These conditions may be treated or prevented by application of devices according to the invention to infected or at-risk skin, to remove target biotoxins, viruses, microbes or their components. Methods of protection against wound infections are also contemplated; by removing target viruses, microbes or microbial agents from the skin on or around a wound, or the site of a future wound (such as in surgery), colonisation of a wound by opportunistic infectious agents can be prevented.

It is also contemplated that targets for devices as described can be those present inside body cavities. For example, a target biotoxin, virus, microbe or microbial component may be causing dysbiosis, disease or disorders of the oral cavity, such as gingivitis, periodontitis, dental caries, or halitosis (bad breath). The target biotoxin, virus, microbe or microbial component may be causing vaginal infections. Devices according to the invention can be applied in and around such cavities to treat or prevent infections.

Agents involved in causing sexually transmitted diseases which could be targeted by devices according to the present invention include Neisseria gonorrhoeae, Chlamydia trachomatis, Treponema pallidum, Ureaplasma urealyticum, and Haemophilus ducreyi.

Agents involved in causing eye infections which could be targeted by devices according to the present invention include Staphylococcus spp., Neisseria gonorrhoeae, and Chlamydia trachomatis.

Agents involved in causing upper respiratory tract infections which could be targeted by devices according to the present invention include Aspergillus spp., Streptococcus pneumoniae and other Streptococcus spp., Pseudomonas aeruginosa, Bordetella pertussiss, Moraxella catarrhalis, Mycoplasma pneumoniae, Mycobacterium tuberculosis, Coxiella burnetii, Klebsiella pneumoniae, Staphylococcus aureus, Legionella pneumophila and proteobacteria such as species of Escherichia, Proteus, and Serratia. Haemophilus influenzae, influenza virus, rhinoviruses and coronaviruses like SARS, MERS and the pandemic SARS-CoV-2.

Devices according to the present invention are also contemplated for use in the removal of biofilms from target surfaces, that is, conglomerations of microbes which adhere to one another and to a surface. Such biofilms can be difficult to remove by conventional means due to the number of organisms and the presence of extracellular factors which can protect the microbes from attack.

Also contemplated are devices as described which are used to deliver useful, commensal, probiotic, non-harmful and symbiotic bacteria and/or microbial components and/or controlled doses of biotoxins for therapeutic and cosmetic purposes between surfaces, including skin. In such cases, a binding agent which can bind a probiotic target is attached to a carrier material and used to bind a multilayer of probiotic microbes or microbial components for release onto selected surfaces. In this way, the devices can be used to collect and/or concentrate beneficial microbes (such as commensals and probiotic organisms) and transfer, transplant, and/or deliver these to another site or surface, including internal delivery such as into the gastrointestinal tract. While the transferred microbes themselves may be bound to the device of the invention by binding agents, replication may still occur, and later-produced microbes may not be bound and may be free to transfer to the target surface. Similar approaches can allow for the collection and/or storage of beneficial bacteria for later use. For this or other purposes (such as forensic analysis or laboratory use) it may even be possible to detach bound biotoxins, viruses, microbes or microbial components from the device, for example by use of a buffer solution by changing pH, or ionic strength, or by using a weak acid. Monosaccharide or disaccharide solutions could also be used to disrupt sugar-protein interactions and thereby release bound biotoxins, viruses, microbes or microbial components.

Further, devices according to the invention can also be used in the trapping or removal of targets which are not strictly biotoxins, viruses, microbes or microbial components, if they can be bound by binding agents as considered herein. For example, allergens and other microscopic components produced by non-microbial life may have surface components such as lectins or glycoconjugates which could be bound by binding agents as described. Examples include plant pollen, fungal spores, dust mite faeces and other components, potential allergens from food such as nuts and shellfish, animal or plant venoms or poisons, and animal products such as dander. Such targets can be harmful to humans or animals, by causing allergic reaction or otherwise. Since allergic reactions are often mediated by cell-surface interactions, and some allergens comprise or consist of glycoconjugates, devices according to the invention can be effective in binding such targets. Advantageously, this could allow for the removal of such targets from surfaces, liquids or gases, or a subject's face or skin. For example, a device prepared in order to provide binding sites for plant pollens could be used to remove pollens from surfaces or a human or animal's eyes or skin.

In an epidemic or pandemic context, the present invention may have various uses, including but not limited to: the decontamination of exposed skin to reduce the risk of transfer during removal of personal protective equipment (PPE); sampling and clean-up of the PPE itself; and the collection of samples from public (for example during screening in transportation hubs/vehicles) to aid in future decision making (imposition of lockdowns, exclusion of public access and transportation units etc.)

The provision of devices for delivering inhibitory compounds/anti-attachment molecules (such as anti-bacterials, antivirals, antimycotics, antitoxins) is also contemplated.

In a laboratory context or elsewhere, another possible use for embodiments of the invention is in the purification of fractions during biopharmaceutical and pharmaceutical processes to capture glycan- and lectin-containing components. For example, this could involve the removal of LPS/endotoxin and microbial residues and contaminants or non-product fractions produced during manufacturing process. This could also apply to recombinant protein/vaccine production to remove host components and enrich a desired product.

Devices and methods of the present invention can also be used in conjunction with surgical gloves or other surgical or medical equipment, for example the addition of devices according to the invention to the surface of surgical gloves. This would act to reduce the possibility of spreading infections during surgery or other care, which is the main source of implanted device bio-contamination during surgeries. An example is the use of surgical gloves during catheter implantation.

### EXAMPLES

The following non-limiting examples indicate some embodiments of the invention.

### Example 1—Device Manufacture

To produce active aldehyde groups for the subsequent linkage of binding agents on a cellulose chain, a periodate oxidation reaction was carried out. The cellulose backbone of 33 gms 100% cotton material was chemically treated with sodium periodate (Sigma-Aldrich 311448) by immersion in solutions of sodium periodate in 0.1M acetate buffer (ratio 1:50, w/v), at a concentration of 5.0 mg/ml. The mixture was kept in the absence of light for efficient reaction, at room temperature, with gentle shaking at 50 rpm for 6 hours. This reaction is thought to take place between the C2-C3 bond of the glucopyranoside ring, and to lead to the formation of two aldehyde groups at the C2 and C3 positions. The resulting compound is the 2, 3 dialdehyde cellulose (DAC)

Afterwards, the material was thoroughly washed with ice-cold distilled water (3 washes, 10 times the absorbed volume each time) to remove the periodate oxidant from the treated materials. For addition of the binding agent (the lectin, glycoprotein, glycoconjugate or neoglycoconjugate) after chemical treatment, it is believed that the active components chemically attach to the DAC residue of the cellulosic backbone. A binding agent solution (1 mg/ml) was prepared by dissolving in PBS at pH 7.4. The above mentioned treated cotton material was immersed into the solution of protein at a ratio of 1:25 (w/v). The materials are incubated for 16 h at 4° C., and then washed in PBS at pH 7.4 for three times in 10 times the absorbed volume.

### Example 2—Biothreat Pathogens

Biothreat bacteria (Francisella tularensis, Clostridium botulinum and Bacillus anthracis (cells and spores), were recovered for growth into stationary phase, and staining of the biothreat targets was performed. After careful analysis of binding data, generated by glycan microarray as previously described, and based on comparison with model organisms, glycoproteins/glycoconjugates and lectins were nominated for the preparation of anti-bacterial cellulose-based devices. Testing of the efficacy of active cellulose-based wipes (produced according to Example 1) was performed in comparison to dry wipes and wipes treated with relevant buffers (dH₂O, PBS), on plastic/metal/glass surfaces contaminated with the above biothreat agents.

Contaminants were prepared from overnight cultures of OD₆₀₀ of 2.0 stained with 0.5% crystal violet. 100 μl of each contaminant was placed per selected test area of 5 cm diameter and dried for 60 min. For each of the following experiments, the wipe was placed in the middle of the contaminated area and was left for 10 minutes without movement. After removal of the wipes, ‘leftover contaminants’ on each surface were recovered by washing with 0.5 ml of PBS pH 7.4 and analysis was performed using a range of quantitative methods for the enumeration of bacteria remaining on the surface (measurement of optical density, colony count, PCR).

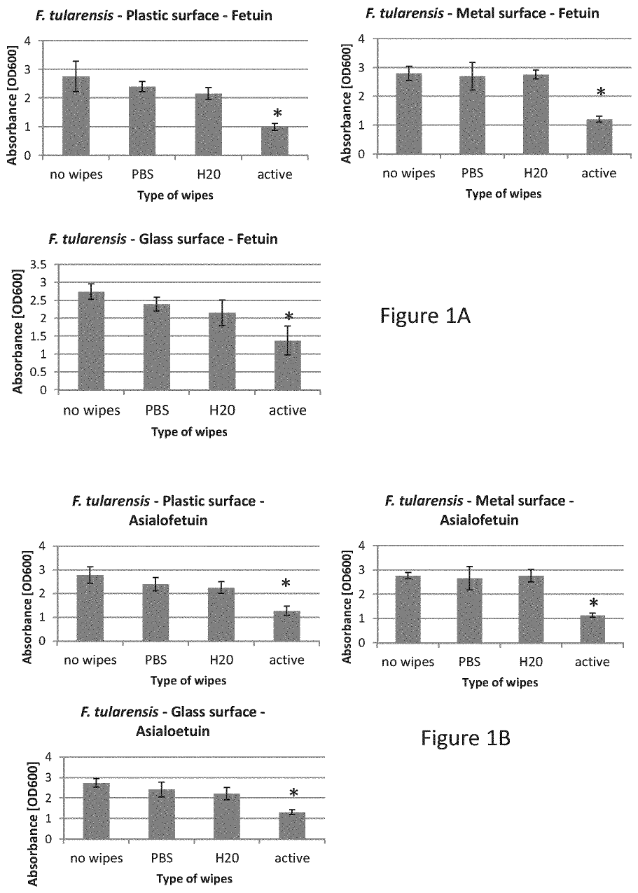

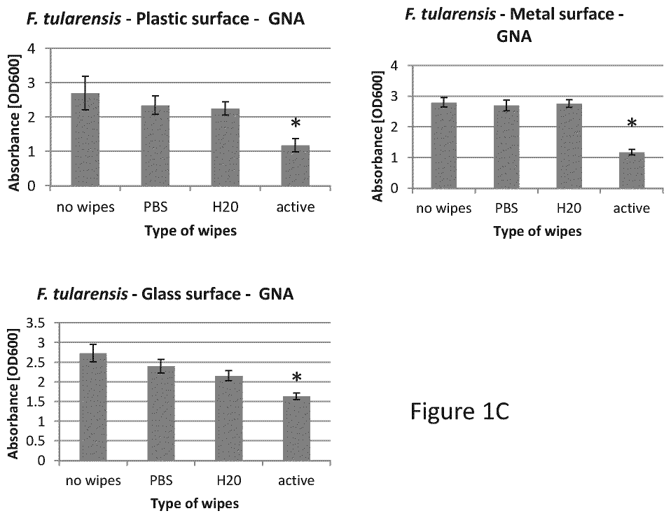

For Francisella tularensis (FIG. 1), efficacy tests were conducted as described above using active wipes comprising fetuin glycoprotein (FIG. 1A), asialofetuin glycoprotein (FIG. 1B) or the lectin GNA (FIG. 1C). Leftover contaminants were monitored by adding a recovery solution of an appropriate buffer and the measurement of absorbance/optical density at 600 nm (OD600). Raw values without surface adjustment are shown. Asterisks indicate statistically different data points in comparison with the ‘no wipes’ condition, based on Student's t-test (p<0.05). Error bars represent standard deviation for triplicate experiments. It can be seen that in all cases, use of ‘active’ wipes treated with the glycoprotein or lectin result in significantly less residual bacteria.

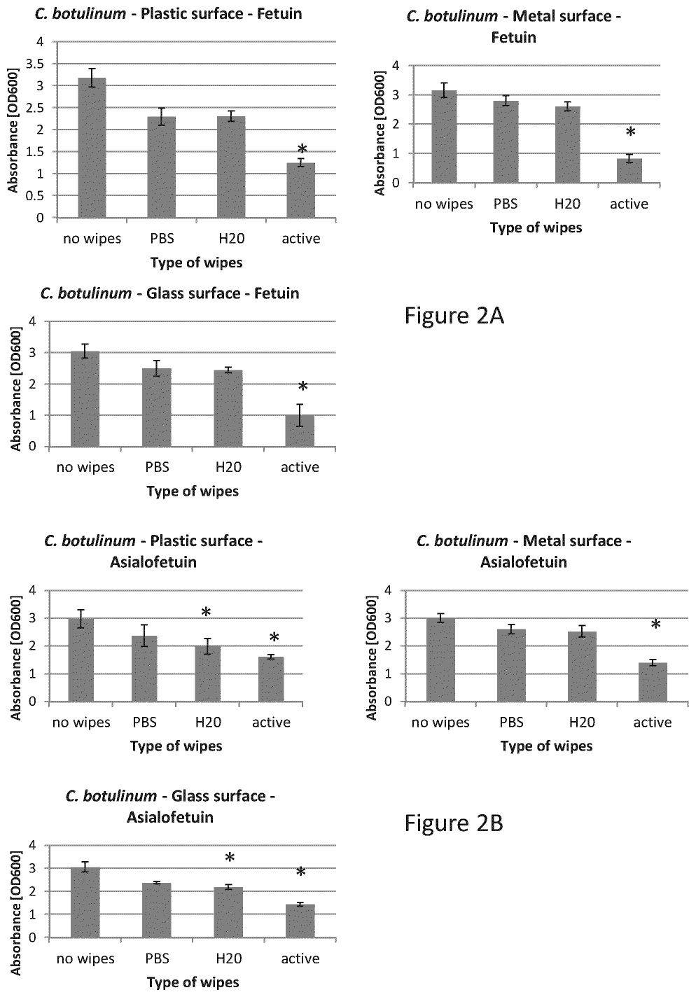

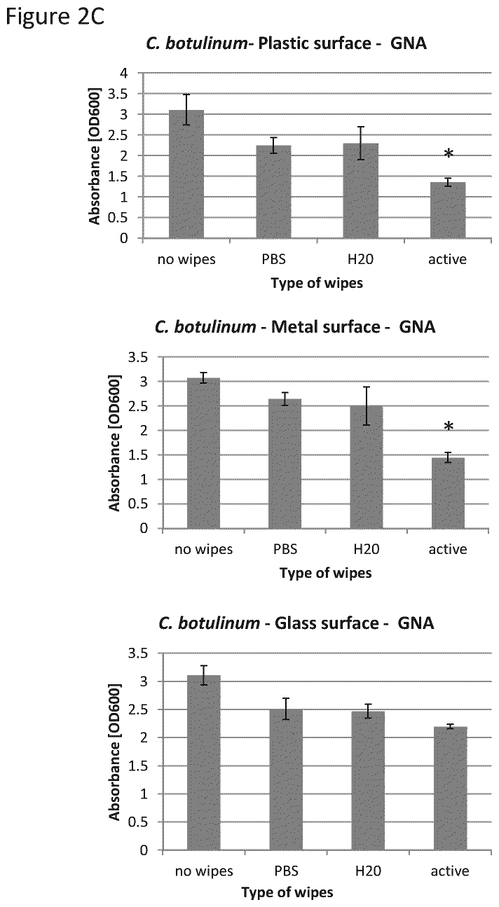

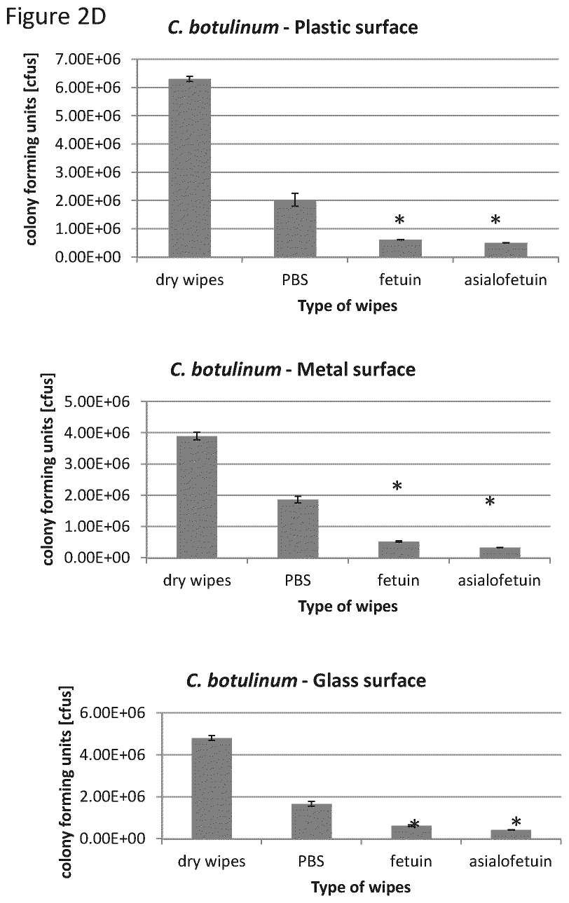

For Clostridium botulinum (FIG. 2) efficacy tests were conducted using active wipes comprising fetuin glycoprotein (FIG. 2A), asialofetuin glycoprotein (FIG. 2B) or the lectin GNA (FIG. 2C). Leftover contaminants were monitored by adding a recovery solution of an appropriate buffer and the measurement of absorbance/optical density at 600 nm (OD600). Raw values without surface adjustment are shown. Asterisks indicate statistically different data points in comparison with the ‘no wipes’ condition, based on Student's t-test (p<0.05). Error bars represent standard deviation for triplicate experiments. It can be seen that in all but one case, use of ‘active’ wipes treated with the glycoprotein or lectin result in significantly less residual bacteria. FIG. 2D shows the leftover bacteria present on the surface after the wipe treatment as measured by the recovery of colony forming units (cfu), against a reference stock of 7.32×10⁸ cfu/ml. For this, recovered contaminants were serially diluted and 100 μl of each was spread on agar plates to grow bacteria. Plates with 30-300 colonies were considered an appropriate range for calculations.

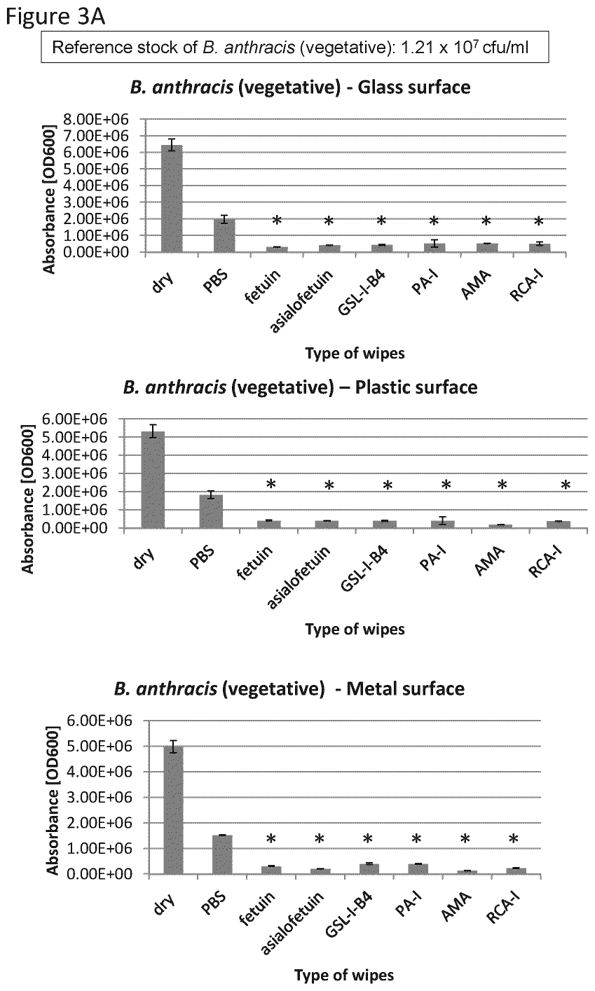

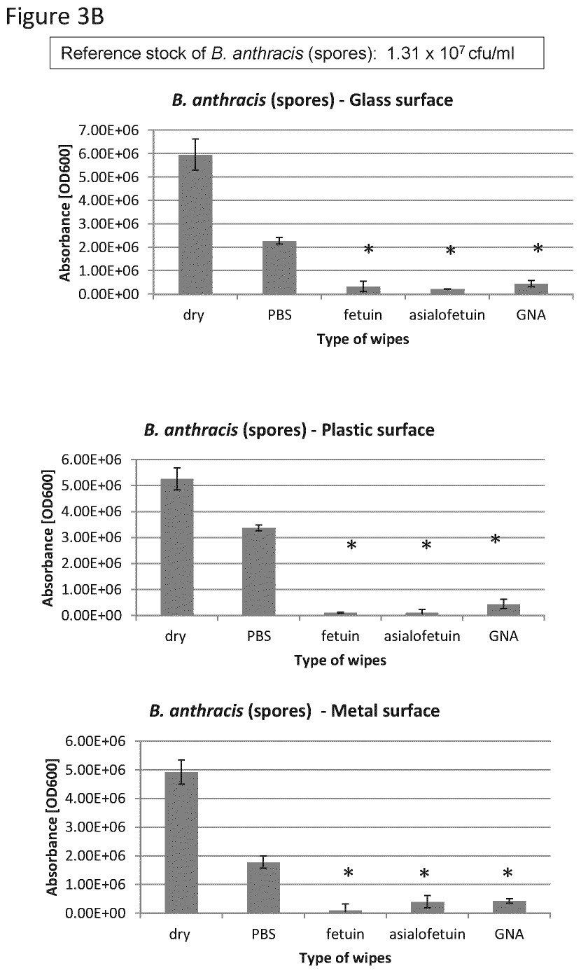

For Bacillus anthracis (FIG. 3 and Table 4) efficacy tests were conducted using active wipes comprising fetuin glycoprotein, asialofetuin glycoprotein, GNA lectin, GSL-I-B4 lectin, PA-I lectin, AMA lectin, or RCA-1 lectin; against B. anthracis cells (FIG. 3A) or spores (FIG. 3B). Leftover contaminants after wipe treatment were measured by the recovery of colony forming units (cfu), against a reference stock of 1.21×10⁷ cfu/ml (vegetative cells) or 1.31×10⁷ cfu/ml (spores). Recovered contaminants were serially diluted and 100 μl of each was spread on agar plates to grow bacteria. Plates with 30-300 colonies were considered an appropriate range for calculations. Raw values without surface adjustment are shown. Asterisks indicate statistically different data points in comparison with surface with no wiping, based on Student's t-test (p<0.05). Error bars represent standard deviation for triplicate experiments. These data are also shown in Table 4, which indicates the percentage of residual contaminant found, compared with a dry wipe treated surface (FET—fetuin, ASF—asialofetuin).

TABLE 4

| | | | | | | | | |
|---|---|---|---|---|---|---|---|---|
| | | | | | GSL-I- | | | |
| Dry | PBS | FET | ASF | GNA | B4 | PA-I | AMA | RCA-1 |

Leftover contamination after using control
and active wipes - B. anthracis vegetative cells

| | | | | | | | | | |
|---|---|---|---|---|---|---|---|---|---|
| Plastic | 100% | 35% | 8% | 8% | N/A | 8% | 8% | 2.5% | 8% |
| surface | | | | | | | | | |
| Glass | 100% | 32% | 7% | 8% | N/A | 7% | 7% | 7% | 7% |
| surface | | | | | | | | | |
| Metal | 100% | 28% | 8% | 1.3% | N/A | 8% | 8% | 1.3% | 2.7% |
| surface | | | | | | | | | |

Leftover contamination after using control
and active wipes - B. anthracis vegetative cells

| | | | | | | | | | |
|---|---|---|---|---|---|---|---|---|---|
| Plastic | 100% | 65% | 1.3% | 1.3% | 8% | N/A | N/A | N/A | N/A |
| surface | | | | | | | | | |
| Glass | 100% | 37% | 5% | 2.3% | 7% | N/A | N/A | N/A | N/A |
| surface | | | | | | | | | |
| Metal | 100% | 32% | 1.3% | 8% | 7% | N/A | N/A | N/A | N/A |
| surface | | | | | | | | | |

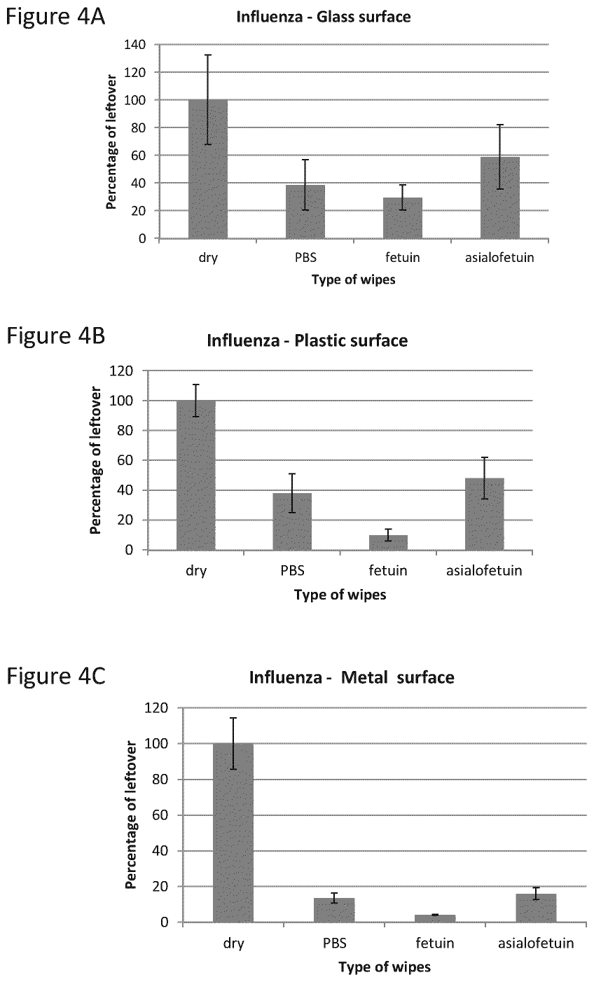

Devices according to the present invention were also used against influenza virus contamination on glass, plastic and metal surfaces. Briefly, the surfaces were contaminated by 500 μl of viral solution, the supernatant being spread onto the surface and allowed to dry for 3 hours. Wipes with attached fetuin glycoprotein, or attached asialofetuin glycoprotein were used. All surfaces were contaminated in triplicate for each type of wipe. For wipe testing, the wipe was placed in the middle of the contaminated area (without any movement). The wipes were left to interact for 10 minutes. After incubation, the wipes were removed and the contaminated area was assessed for the recovery of leftover. Each surface was washed by 1 ml of PBS and the recovery solution was transferred to sterile Eppendorf tube for viral RNA isolation and quantification of leftover from glass (FIG. 4A), plastic (FIG. 4B) and metal (FIG. 4C). The figures show virus amount isolated from the surface, relative to that found after use of a dry wipe only. Error bars represent standard deviation, all in triplicates. The results show that while fetuin-linked wipes appeared to reduce residual virus, leaving only 2-28% of viral particles after static capture (see Table 5), asialofetuin-linked wipes did not appear to have any effect compared to that of PBS-treated wipes.

TABLE 5

| | Dry | PBS | Fetuin | Asialofetuin |
|---|---|---|---|---|
| Glass surface | 100% | 39% | 28% | 58% |
| Plastic surface | 100% | 40% | 11% | 48% |
| Metal surface | 100% | 12% | 2% | 13% |

### Example 3—Foodborne Pathogens

Devices according to the present invention (produced according to Example 1) were also used against bacteria associated with foodborne disease, on glass, plastic and metal surfaces. Table 6 shows the bacteria tested (EHEC E. coli O157:H7 and Enterobacter cloacae), and the lectin which was bound to the carrier material in each case.

TABLE 6

| Target | Model target (contaminant) | Lectin wipe |
|---|---|---|
| Coliforms | SYTO82 EHEC E. coli O157:H7 | WGA |
| | SYTO82 Enterobacter cloacae | GSI-B4 |

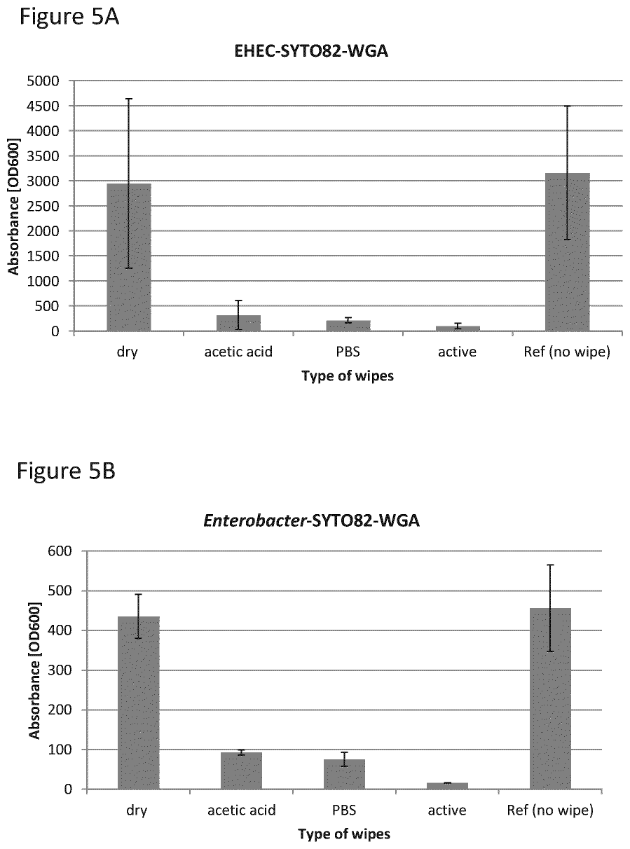

FIGS. 5A and 5B show the results of efficacy tests on these bacteria. As in the preceding example, contaminants were prepared from overnight cultures stained with crystal violet. 100 μl of each contaminant was placed per selected test area and dried for 60 min. For each of the following experiments, the wipe was placed in the middle of the contaminated area and was left there for 10 minutes without movement. Residual contaminants were monitored by measuring fluorescence after staining for SYTO82 (FIGS. 5A, 5B). Raw values without surface adjustment are shown. Error bars represent standard deviation from triplicate experiments. For reference, a contaminated surface which was not wiped was used. Based on measurement of leftover fluorescence after 10 min static test active wipe with WGA lectin left only 1% of E. coli O157:H7, while active wipe with GSI-B4 left 4% of Enterobacter cloacae. The results, indicating the efficacy of control and active wipes are also shown in Table 7.

TABLE 7
percentage of contamination compared to unwiped surface

| | Wipe condition | | | |
|---|---|---|---|---|
| Contaminant and fluorescence | dry | acetic acid | PBS | active |
| SYTO82 EHEC E. coli O157:H7 | 93 | 10 | 5 | 1 |
| SYTO82 Enterobacter cloacae | 102 | 22 | 19 | 4 |

### Example 4—Other Skin Agents (Bacterial/Fungal)

Other microbes which are known to colonise the skin are potential targets for devices according to the present invention. These include Propionibacterium spp., Malassezia spp. (formerly known as Pityrosporum), Candida spp., Aspergillus spp., Staphylococcus spp., Streptococcus spp., Pseudomonas spp., and Haemophilus influenzae. To illustrate, example devices (produced according to Example 1) were also used against the bacterium Propionibacterium acnes and the fungus Candida albicans, which are associated with skin diseases and disorders.

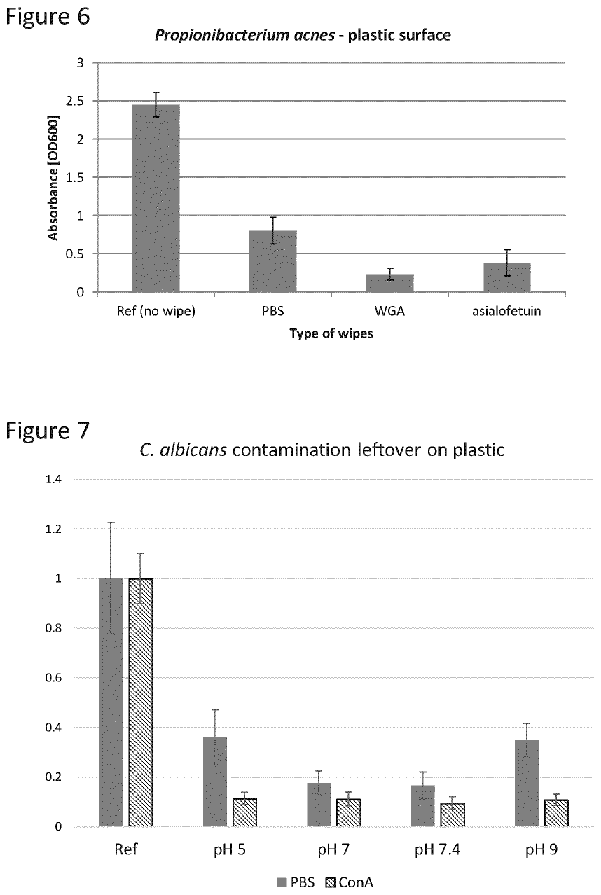

Wipes coupled to the glycoprotein asialofetuin (ASF) or the lectin WGA were used against plastic surfaces contaminated with Propionibacterium acnes as in the preceding examples (FIG. 6). After the static wipe test was performed, for 10 minutes, again as in the preceding examples, 33% leftover contaminants were observed for PBS wipes, 15.8% for ASF wipe and only 9.5% for WGA wipe showing that WGA wipe was able to capture 90.5% of contaminants by contact only (no movement).

Wipes coupled to the lectin ConA were used against plastic surfaces contaminated with C. albicans (FIG. 7), with contamination and wipe tests as previously described. To compare capturing efficacy at various pH levels, the wipes were prepared using buffers with pH of 5, 7 and 9 and standard pH of 7.4; with or without the active component (ConA). The effect of capturing by the active component was observed to be similar over a wide range of pHs from 5 to 9. Thus, the wipes had comparable efficacy under the selected conditions and pH alteration did not influence the capabilities of the active component.

### Example 5—Wound Decontamination and Care

To evaluate the device and its efficacy for capture from biological surfaces a pig skin model was used. During the last 20 years, pig skin has been the predominant human skin model used for human skin disease research. The reasoning behind this is based on the similarities between the anatomies of the two species, in comparison to any other laboratory animal. For example, the dermal collagen of pigs is more similar to humans than any other common laboratory animal. Pig skin has been well established and studied for its use for human disease wound care and infection studies. The epidermal thickness and structure of pig skin possesses strong similarities to human skin. The blood vessel features, and types of hair follicles are highly related. Strains of E. coli and C. albicans were used as a target organism.

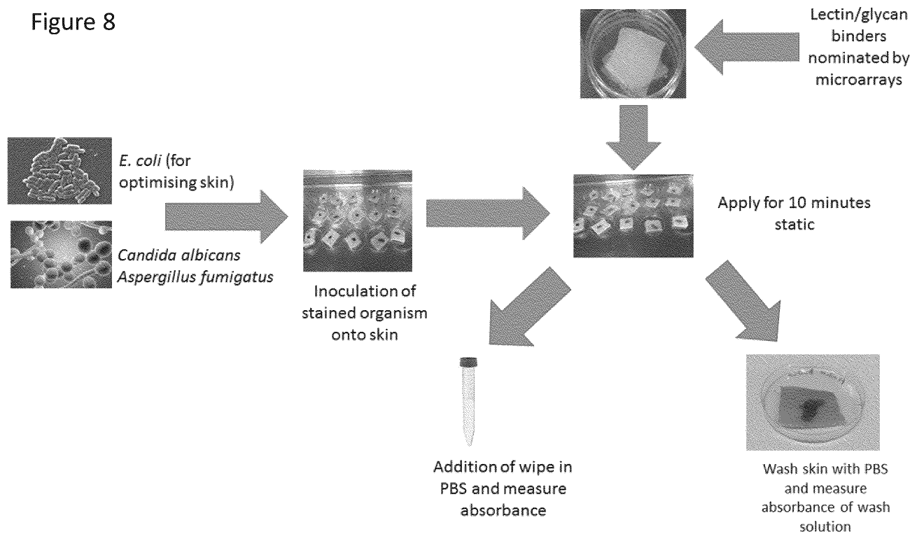

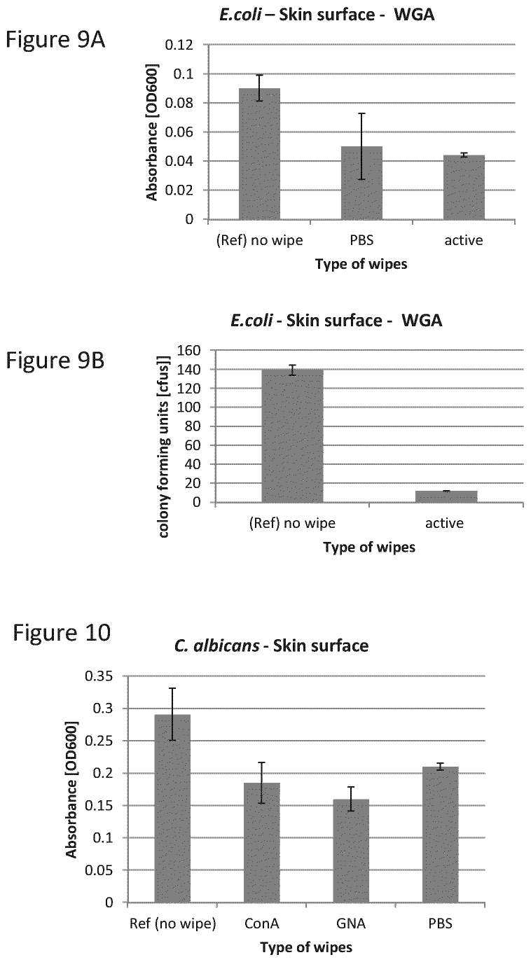

The method is illustrated in FIG. 8. Briefly, to eliminate natural contaminants of porcine skin, section samples were placed into water at 60° C. for 30 seconds. 100 μl of either E. coli (stained with crystal violet) or Candia albicans (stained with Trypan Blue fungal stain) culture of OD 2.0 was pipetted onto each porcine sample and left to dry on the skin sample for 30 minutes. The wipe capturing tests were carried out by static contact for 10 minutes as previously described using devices produced according to Example 1, and then 1 ml of LB or yeast medium was added onto each of the porcine skin samples and contaminant mix was recovered. This mix was monitored for growth to quantify leftover E. coli (FIGS. 9A and B) and C. albicans (FIG. 10). FIGS. 9A and 9B shows the E. coli recovered from the pig skin after treatment with cotton-based wipe capture tests, where the active wipe contained the WGA lectin, as measured by absorbance at 595 nm wavelength (FIG. 9A) or by colony forming units (FIG. 9B). FIG. 10 shows the C. albicans DSM 6659 recovered from the pig skin after treatment with cotton-based wipe capture tests, where the active wipe contained the ConA lectin or the GNA lectin, as measured by absorbance after a 15 hour growth period.

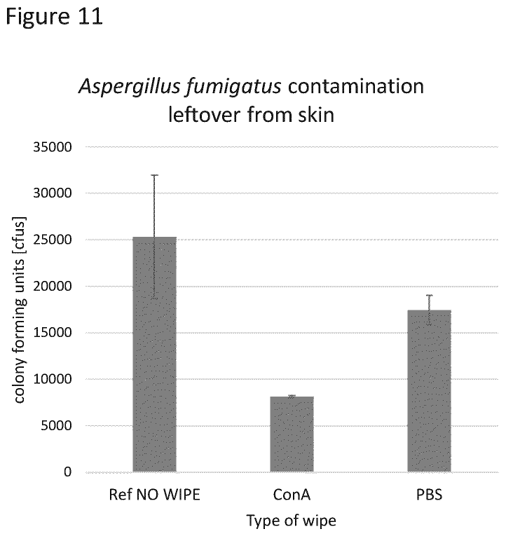

Wipes coupled to the lectin ConA were used against porcine skin sections contaminated with Aspergillus fumigatus, with contamination protocols and wipe tests as previously described. A. fumigatus was recovered from the pig skin after treatment with cotton-based wipe capture tests, where the active wipe contained the ConA lectin, as measured by colony forming units after plating 1:10 serial dilutions of leftover contaminants onto Potato-Dextrose Agar (FIG. 11). Colony count analysis was performed after 24 h incubation at 30° C. FIG. 11 shows the A. fumigatus Fresenius 819 strain recovered from the pig skin after treatment with cotton-based wipe capture tests, where the active wipe contained the ConA lectin.

1. A device comprising:
a carrier material comprising a carbohydrate-based polymer; and
a binding agent;
wherein:
the binding agent is attached by one or more covalent bonds to the carrier material; and
the binding agent can bind to a target, the target being one or more of a biotoxin, a virus, an allergen, a microbe, and a microbial component.

2. A device according to claim 1, wherein the carrier material comprises cellulose.

3. A device according to claim 1, wherein the carrier material comprises one or more of cotton and paper.

4. A device according to claim 1, comprising a fluid in which the carrier material is dissolved, suspended, dispersed, emulsified, or otherwise carried.

5. A device according to claim 2, wherein the binding agent is attached to the cellulose by one or more covalent bonds.

6. A device according to claim 1, wherein the binding agent comprises one or more of an antithrombotic agent, an anti-inflammatory, an antibody, an antigen, an adhesin, an immunoglobulin, an enzyme, a hormone, a neurotransmitter, a cytokine, a protein, a globular protein, a cell attachment protein, a peptide, a cell attachment peptide, a proteoglycan, a toxin, a polysaccharide, a carbohydrate, a fatty acid, a drug, a vitamin, a DNA segment, a RNA segment, a nucleic acid, a dye, and a ligand.

7. A device according to claim 1, wherein the binding agent comprises one or more of a lectin, a glycoprotein, an oligosaccharide, and a glycoconjugate.

8. (canceled)

9. A device according to claim 7, wherein the binding agent comprises a lectin selected from one or more of AIA/Jacalin, RPbAI, AAL, ABL, ACA, AMA, BPA, CAA, Calsepa, CCA, ConA, CPA, DBA, DSA, ECA, EEA, GHA, GNA, GSL-I-B4, GSL-II, HHA, HPA, Lch-A, Lch-B, LEL, LTA, MAA, MOA, MPA, NPA, PA-I, PCA, PHA-E, PHA-L, PNA, PSA, RCA-I/120, SBA, SJA, SNA-I, SNA-II, STA, UEA-I, VRA, VVA-B4, WFA, and WGA.

10. (canceled)

11. A device according to claim 7, wherein the binding agent comprises a glycoprotein.

12. A device according to claim 11, wherein the glycoprotein is one or more of Uromodulin (Tamm-Horsfall protein), Fetuin, Asialofetuin, Invertase, Fibrinogen, alpha-1-antitrypsin, α-Crystallin, Ceruloplasmin, alpha-1-acid glycoprotein, RNAse B, Transferrin, β-lactoglobulin, C.-lactalbumin, Albumin, B-casein, C-casein, K-casein, Lactoferrin, Ovalbumin, Ovomucoid, Ovotransferrin, and derivative glycomacropeptides.

13. A device according to claim 12, wherein the glycoprotein is one or more of Lactoferrin, Fetuin, Asialofetuin, and α-Crystallin.

14. (canceled)

15. A device according to claim 7, wherein the binding agent is a glycoconjugate or neoglycoconjugate selected from one or more of Blood Group A-BSA, Blood Group B-HSA, Fuc-α-4AP-BSA, Fuc-β-4AP-BSA, 2′Fucosyllactose-BSA, Difucosyl-para-lacto-N-hexaose-APD-HSA (Lea/Lex), Tri-fucosyl-Ley-heptasaccharide-APE-HSA, Monofucosyl, monosialyllacto-N-neohexaose-APD-HSA, Gal-β-4AP-BSA, Galα1,3Gal-BSA, Gal-α-1,3Galb1, Gal-β-1,4Gal-BSA, Gal-α-1,2Gal-BSA, 4GlcNAc-HSA, Gal-α-PITC-BSA, Gal-β-ITC-BSA, Glc-β-4AP-BSA, Glc-β-ITC-BSA, GlcNAc-BSA, Globotriose-HSA, Globo-N-tetraose-APD-HSA, Globotriose-APD-HSA, GM1-pentasaccharide-APD-HSA, Asialo-GM1-tetrasaccharide-APD-HSA, Globo-N-tetraose-APD-HSA, Globotriose-APD-HSA, H Type II-APE-BSA, H-Type 2-APE-HSA, Man-α-1,3(Man-α-1,6), Man-BSA, Man-α-ITC-BSA, Man-b-4AP-BSA, LacNAc-BSA, LacNAc-α-4AP-BSA, LacNAc-β-4AP-BSA, Lac-β-4AP-BSA, Lacto-N-tetraose-APD-HSA, Lacto-N-fucopentaose I-BSA, Lacto-N-neotetraose-APD-HSA, Lacto-N-fucopentaose II-BSA, Lacto-N-fucopentaose III-BSA, Lacto-N-difucohexaose I-BSA, Lewis a-BSA, Lewis x-BSA, Lewis y-tetrasaccharide-APE-HSA, LNDI-BSA/Lewis b-BSA, Di-Lex-APE-BSA, Di-Lewisx-APE-HSA, Tri-Lex-APE-HSA, L-Rhamnose-Sp14-BSA, 3′-Sialyllactose-APD-HSA, 3′Sialyl-3-fucosyllactose-BSA, 6′-Sialyllactose-APD-HSA, Xyl-α-4AP-BSA, Xyl-β-4AP-BSA, 3′Sialyl Lewis x-BSA, 3′Sialyl Lewis a-BSA, 6-Sulfo Lewis x-BSA, 6-Sulfo Lewis a-BSA, 3-Sulfo Lewis a-BSA, 3-Sulfo Lewis x-BSA, Sialyl-LNF V-APD-HSA, and Sialyl-LNnT-penta-APD-HSA.

16. A device according to claim 7, wherein the binding agent is a glycoconjugate, neoglycoconjugate or a glycoprotein, and has terminal residues comprising one or more of mannose, N-Acetylglucosamine, N-Acetylgalactosamine, N-acetylneuraminic and N-glycolylneuraminic acid (sialic acid), Galactose, Glucose, and Fucose moieties.

17. (canceled)

18. A device according to claim 1, wherein the device further comprises an antimicrobial substance selected from one or more of an antiseptic, an antibiotic, a detergent, silver, copper, or EDTA.

19. (canceled)

20. A device according to claim 1, wherein the carrier material is in the form of a cloth, a wipe, a wound dressing, a swab, a filter, a pad, a blanket, a mat, a mask, or a coating.

21. A method of removing a biotoxin, a virus, an allergen, a microbe, and/or a microbial component from a surface, gas, or liquid, the method comprising:
providing a device according to claim 1; and
contacting the surface with the device, or passing gas or liquid through the device.

22. (canceled)

23. A method according to claim 21, wherein the binding agent binds to the biotoxin, the virus, the allergen, the microbe, and/or the microbial component to be removed.

24. A method according to claim 21, wherein the biotoxin, the virus, the allergen, the microbe, and/or the microbial component is a spore.

25. A process for making a device, the method comprising:
providing a carrier material comprising a carbohydrate-based polymer;
treating the carrier with an oxidising agent to produce acid and/or aldehyde groups; and
contacting the treated carrier with a binding agent which comprises one or more of a lectin, a glycoprotein, a glycoconjugate, and their derivatives, such that the binding agent is linked to the carbohydrate-based polymer by one or more covalent bonds.

26. (canceled)

27. (canceled)

28. A process according to claim 25, wherein the oxidising agent is selected from the group comprising:
a periodate;
sodium periodate;
2,2,6,6-tetramethylpiperidine-1-oxyl radical (TEMPO);
sodium nitrate or sodium nitrate in phosphoric acid;
activating agent tosylchloride in the presence of organic solvent and a base;
and combinations thereof.

29. (canceled)

30. A device according to claim 1, wherein the carrier material further comprises a buffer.
